(19) United States
(12) Patent Application Publication
(10) Pub. No.: US 2020/0096524 A1
(43) Pub. Date: Mar. 26, 2020

(54) PLASMA AND LIVER LIPID SPECIES AS BIOMARKERS OF FATTY LIVER

(71) Applicant: École Polytechnique Fédérale de Lausanne (EPFL), Lausanne (CH)

(72) Inventors: Pooja JHA, Chavannes-Renens (CH); Johan AUWERX, Buchillon (CH)

(21) Appl. No.: 16/483,576

(22) PCT Filed: Feb. 5, 2018
(86) PCT No.: PCT/EP2018/052815

§ 371 Date: Aug. 5, 2019

(30) Foreign Application Priority Data
Feb. 3, 2017 (EP) 17154690.6

(51) Int. Cl. G01N 33/92

(52) U.S. Cl. CPC G01N 33/92; G01N 2800/08; G01N 2405/02; G01N 2800/52; G01N 2800/50

(57) ABSTRACT

The invention relates to plasma or liver lipid species selected from the triacylglycerol (TAG) and cardiolipin (CL) lipid classes measured by LC-MS/MS and use thereof as diagnostic and prognostic biomarkers of fatty liver, as well as to monitor the efficacy of preventive and therapeutic measures to lower liver fat content.

### FIELD OF THE INVENTION

The invention relates to (i) seven plasma triglyceride (TAG) species and/or (ii) seven liver triglyceride (TAG) and eight liver cardiolipin/monolysocardiolipin species measured by targeted LC-MS and use thereof as diagnostic and prognostic biomarkers of fatty liver, as well as to monitor the efficacy of preventive and therapeutic measures to lower liver fat content.

### BACKGROUND OF THE INVENTION

Fatty liver is characterized by excess of fat/triglyceride accumulation in the liver. It is the common characteristic feature of most liver diseases including non-alcoholic fatty liver disease (NAFLD) and alcoholic fatty liver disease (AFLD). Both NAFLD and AFLD encompasses a spectrum of liver abnormality which includes fatty liver/steatosis, steatohepatitis, fibrosis and cirrhosis. The major causes of NAFLD are obesity, diabetes, hyperlipidemia, rapid weight loss, genetic inheritance and side effect of certain medications, including aspirin, steroids, tamoxifen, and tetracycline. Whereas AFLD is induced by excess intake of alcohol. While the disease aetiology is different for NAFLD and AFLD, the underlying abnormality of excess fat accumulation in the liver or fatty liver is common. The current diagnosis of these diseases relies on invasive liver biopsies, which are discretionary in nature due to the histological scoring involved and may result in “under-” or “mis-diagnosis” of these diseases and often a late start of treatment. Thus there is a need to develop further methods that are easy to carry out, safe, accurate and reliable for diagnosing liver diseases and for monitoring the efficacy of preventive and therapeutic measures to lower liver fat content.

### SUMMARY OF THE INVENTION

In the present invention, MS-based lipidomics in plasma and liver have been used, combined with phenomics from 50 BxD genetic reference population strains, to identify biomarkers of fatty liver. The MS approach was standardized to capture triglyceride (TAG) species and cardiolipin (CL) species abundant in plasma and liver of all the genetically diverse 50 BXD strains, which enhanced the scope of these species as biomarkers to different degrees of fatty liver. These five beneficial (CL(LLLL), MLCL(LLL), TAG(52:5), TAG(52:4) and TAG(54:6)) and ten harmful (MLCL(LOO), MLCL(LLO), CL(LOOPo), CL(LLPoP), CL(LOOO), CL(OOOP), TAG(52:2), TAG(54:3), TAG(56:3), TAG(50:2)) CL and TAG biomarkers reflect carefully their levels in liver and plasma and correlate with other biological indicators of liver fat accumulation. Importantly, the beneficial lipid species correlate negatively and the harmful species correlate positively with obesity and NAFLD traits. They are not restricted to a particular diet and therefore can be suitable for the diagnosis and prognosis of individuals with fatty liver resulting from either genetic or different dietary or environmental (e.g. medication, toxins) insults. The above proposed plasma TAG biomarkers are hence easy to apply and non-invasive, less labor intensive and standardized. They also pose risk for adverse effects, associated with liver biopsies. Finally, these plasma biomarkers also offer a chance to monitor disease progression and the efficacy of preventive or therapeutic interventions. The liver TAG, CL and MLCL biomarkers also offer a chance to monitor disease progression and the efficacy of preventive or therapeutic interventions in liver.

In one aspect the invention provides a method for diagnosing a fatty liver disease or a predisposition therefor in a subject, said method comprising
providing a sample derived from a subject suspected to suffer from a fatty liver disease,
determining in the sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
comparing the amount of the at least one plama or liver triglyceride (TAG) biomarker, liver cardiolipin (CL) biomarker and/or monolysocardiolipin (MLCL) biomarker with a reference, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for the fatty liver disease.

In another aspect the invention provides a method for monitoring the progression of a fatty liver disease in a subject diagnosed to suffer from a fatty liver disease, said method comprising
providing a sample derived from a subject diagnosed to suffer from a fatty liver disease,
determining in the sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
comparing the amount of the at least one plasma or liver triglyceride (TAG) biomarker, liver cardiolipin (CL) biomarker and/or monolysocardiolipin (MLCL) biomarker with a reference, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for the fatty liver disease progression.

In a further aspect the invention provides a method for monitoring and/or adapting the efficacy of a therapy for treatment of a fatty liver disease in a subject diagnosed to suffer from a fatty liver disease, said method comprising
a) providing samples derived from a subject before and after the subject undergoes the therapy,
b) determining in the samples of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
c) comparing the amount of the at least one plasma or liver triglyceride (TAG) biomarker, liver cardiolipin (CL) biomarker and/or monolysocardiolipin (MLCL) biomarker after therapy against the amount before therapy, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for inefficacy of the therapy,
d) adapting and/or changing the therapy for treatment of a fatty liver disease in a subject if the therapy is found to be ineffective according to the step c).

In a further aspect the invention provides use of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof, for in-vitro diagnosing fatty liver disease or the predisposition therefor.

In a further aspect the invention provides a plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof for use in diagnosing fatty liver disease or the predisposition therefor in a subject.

### BRIEF DESCRIPTION OF THE FIGURES

FIG. 1 shows an overview of dietary impact on plasma lipidome of BXD strains. (A) Coefficient of variation (CV) in lipid measurements driven by different factors: technical (median CV=4.9%), extraction (10.9%), biological (CV=24.2%), across strain (within diet) (CV=37.8% CD, 40.3% HFD), and across diet (CV=45.5%). The line at the center of the violin plot represents median and the dot, the mean. (B) Heatmap analysis with an unsupervised hierarchical clustering of 129 measured lipid species for each BXD cohort, which shows distinct dietary impact. Lateral panel indicates the diet code, CD is represented in green and HFD in purple. (C) A 2D principal component analysis (PCA) plot of the BXDs shows a clear separation between the CD and the HFD cohorts on PC1 (horizontal) but not PC2 (vertical). Numbers next to the dot corresponds to the BXD strain number. (D) Volcano plot showing the diet effect on all lipid species. The top 10 most significantly different lipid species between the two groups (log 2FC>1.3, p<0.001) are indicated by their names on the plot. Adjusted P.value corresponds to Benjamini-Hochberg corrected significance. (E) Schematic representation of the systemic profile of de-novo lipogenesis (DNL) and monounsaturated fatty acids (MUFA) (top) and polyunsaturated fatty acids (PUFA) (bottom) in the BXDs, based on the free fatty acids (FFAs) level. Only the measured FFAs are shown in the figure. Significant changes (HFD vs. CD; p<0.05) for FFA levels, the desaturases and elongases (calculated as the ratio of the product and precursor FFA) are shown as red for increase or blue for decrease (HFD vs. CD); grey color reflects no significant change. The activity of the enzymes was assessed by the ratio of the product and precursor FFA conversion by the respective enzyme. (F) Two selected lipid species from the above schematic representation. (G) Pie chart showing the dietary enrichment of the 6 most common side chain FAs in all the lipid species measured. The number in the center of the pie chart indicates the total number of lipid species having at least one respective FA side chain. (More than one identical FA side chain in lipid species was not given any weight for the calculation). Red indicates the number of lipid species (having the respective FA side chain) increased in HFD vs. CD. Blue indicates the number of lipid species (having the respective FA side chain) increased in CD vs. HFD. Grey indicates no significant difference between diets. P-value <0.05 by Welch's t-test was considered significant between diets. N=44 (CD), 34 (HFD) BXD strains for all figures hereafter.

FIG. 2 shows unweighted correlation network of lipid species and their link with metabolic phenotypes. (A and B) Spearman correlation network of all lipid species measured in CD (A) and HFD (B). Lipid species are color coded as 10 major lipid classes. A threshold of 1e-5 was used as a best compromise between a very dense and sparse network. (C and D) Heatmap analysis with an unsupervised hierarchical clustering of Spearman's correlation rho value of 38 lipids with metabolic phenotypes. These 38 lipids show the same correlation trend in both diets. The vertical green lipid cluster represents the healthy/positive markers of metabolic health/fitness whereas the vertical red cluster represents the unhealthy/negative markers of metabolic health/fitness. The horizontal green phenotype cluster represents healthy metabolic traits whereas the red cluster are unhealthy metabolic traits. Lipids indicated in blue font in both diets (21 in CD and 12 in HFD) are the top best predictors of metabolic health (they show significant correlation with more than 50% of the metabolic traits in either diet). Red indicates positive correlation and blue negative.

FIG. 3 shows weighted correlation network analysis (WGCNA) identifies lipid modules as predictors of metabolic health. (A) Clustering dendograms and heatmap analysis of modules defined in the WGCNA for CD and HFD. Dendograms are generated using modules eigengenes (ME). Heatmap plots represent the ME adjacencies, where each row and column corresponds to each module, represented by a color and labeled by the main lipid class contained therein (right legend). For each module, the side chain composition and degree of unsaturation are also indicated. Red indicates high ME adjacency (positive correlation) and blue low ME adjacency (negative correlation) as shown in the color legend (left). (B) Color table representing the correspondence of CD- and HFD-specific modules described in panel A. Numbers on the side/below the colors indicate the number of lipid species in each module. Numbers in the table represent the number of lipid species, which are common in both the CD and HFD modules. Grey color represents lipids that were not assigned to any module (background lipids). Color legend (white-red) indicates the negative 10-base logarithmic of the p-value obtained with the Fisher test. (C) Stack bars representing the proportion of FAs in the side-chain of the lipids of each module. MCFA: 10:0 and 12:0; other LCFA: 14:0, 15:0, 16:1, 17:0, 17:1, 18:3, 18:4, 19:0, 19:1, 20:0, 20:1, 20:2, 20:3, 20:5, and 21:0; VLCFA: 22:0, 22:1, 22:4, 22:6, 24:0, 24:1, and 24:6. (D) Module-phenotype correlation. Heatmap representing the correlation of the modules (columns) with selected metabolic phenotypes (rows) in both CD (left) and HFD (right). Modules are represented as explained in panel A. Each cell is color-coded by the Pearson's correlation coefficient according to the legend color on the right. Red cells represents positive correlation, while blue represents negative correlation. Modules and metabolic traits in green font represent healthy modules and traits whereas those in red font represent unhealthy modules and traits respectively. The stars in the cells represent the p-value of the correlation (p<0.05 *, p<0.01**, p<0.001 ***). Module color name is indicated in the legend. Table 2C indicates lipids in each module in both diets.

FIG. 4 shows plasma lipid species are highly heritable in both diets. (A) Dot plot showing heritability/variance of all lipid species. Heritability (h²) was calculated by one-way (CD/HFD) or two-way (Mixed) ANOVA. The variance explained by GxE, diet and unexplained variance (non-dietary, non-genetic) was calculated by two-way ANOVA. Numbers indicated on the graph represent the total number of lipid species that have z 50% of their variance explained by the highlighted factor. The purple line represents median variance explained. (B) h² of the highest and lowest abundant lipid species in each class. The most abundant lipids are indicated as “High” and the least abundant ones as “Low”. Within diet (CD/HFD), the variance explained purely by genetic factors is higher for all the lipids than compared to when dietary cohorts are mixed by strain (CD+HFD). Although many lipids are highly affected by diet (TAG(52:2), DAG(18:1/18:1)), others are not (PC(36:2), CoQ8). GxE and unexplained variance is quite variable for all lipid species. (C) Illustrative example of the levels of 3 lipid species from panel B, having high h² (h²>50%) in both diets but highly affected by diet (TAG(52:2)), or unaffected by diet (PC(20:1/22:6)) or with a minor dietary effect (stearidonic acid). (D) Manhattan plot of lipid species in CD and HFD. The blue dotted line represent the threshold of genome-wide p-value <0.05; the black dotted line represents the threshold of local p-value <0.05. Names of the lipid species with p-value <0.01 are indicated on the plot. Lipid species name followed by “.C” indicates QTL in CD and names followed by “.H” denotes QTL in HFD. TG represents TAG and DG, DAG. (E) Schematic representation of the QTL link to GWAS identified genes for blood lipids and metabolic traits.

FIG. 5 shows identification of lipid species as biomarkers of NAFLD (non-alcoholic fatty liver disease)/fatty liver (A) Plasma and liver TAG concentration is not different between the CD and HFD cohorts (B) Pearson correlation between the serum and liver triglyceride concentration. (C-D) 55 common lipid species between plasma and liver were correlated using Spearman's method. (C) Histogram of the rho correlation value of these 55 lipid pairs in CD (left) and HFD (right). (D) Correlation of rho values between CD and HFD from panels C; i.e. a correlation of correlations from panel C to identify lipids, which behave similarly despite the major dietary switch. The dark green dots indicate the 8 lipid species with significant positive correlation (p<0.05), whereas the red dot indicates one species with significant negative correlation between both liver and plasma and between CD and HFD. Purple dots indicate lipid species with opposite correlation in CD and HFD, reflective of GxE effect. (E) Pearson correlation of 9 lipid species (identified in panel D) in liver and plasma. (F) Corrplot showing the Pearson correlation of the 9 lipids identified in panel D (red and green dots) alongside plasma and liver TAG with selected NAFLD readouts. Lipids in red font indicate pro-NAFLD biomarkers and those in blue font indicate anti-NAFLD biomarkers. n=42-47 for heat map for both CD and HFD.

FIG. 6 shows lipid species as predictors of metabolic traits. (A) Heatmap analysis with an unsupervised hierarchical clustering of spearman's correlation rho of all the lipid species and the metabolic traits. (B) Heatmap analysis with an unsupervised hierarchical clustering of spearman's correlation rho of selected lipid species (which showed maximum correlation with metabolic traits) and metabolic traits. For B, the vertical green lipid cluster represents healthy lipids (specific and common in each diet), whereas the vertical red cluster represents the unhealthy lipids (specific and common in each diet). For A and B the horizontal green phenotype cluster represents healthy metabolic traits whereas the red cluster represents unhealthy metabolic traits. Red indicates positive correlation and blue negative correlation.

FIG. 7 shows weighted correlation network analysis (WGCNA) of plasma lipids. (A) Hierarchical cluster trees of lipid species identified in CD and HFD modules. Each vertical line corresponds to a lipid species and the height is a measure for the dissimilarity based on the topological overlap. Lipids in each module are assigned with the same color, represented in the color band below the dendrograms. Lipids not assigned to any of the modules are colored grey (background). (B) Weighted correlation networks of modules identified in CD and HFD. Main lipid classes of modules are represented.

FIG. 8 shows correlation between h² and QTL and highly significant module QTLs. (A) Spearman correlation between h² and the highest QTL LOD score of each lipid (left) and between h² and the −log 10(P.Value) for the QTL LOD score of each lipid. (B) 4 significant module QTLs (p<0.05) along with their confidence interval and genes in that region in CD (3 QTLs in the green module and 1 in red module). (C) One significant module QTL (p<0.05) along with their confidence interval and genes in that region in HFD (pink module). For B and C: Red dotted line represents the threshold of genome-wide p-value <0.05; the blue dotted line represents the threshold of local p-value <0.05. Gene names are written according to their order in the chromosomal region. Lipids indicated in red font are the know lipid metabolism associated genes. (D) QTL representation of the metabolic hotspot region on chromosome 2 in CD. Lipids with genome-wide significance at this region (99-110 Mb) are represented by complete lines, whereas lipids with local significance are represented by dashed lines. Blue dotted line represents the threshold of local p-value <0.05.

FIG. 9 shows relationship of lipid species QTLs to human GWAS phenotypes. (A) QTL locus for TAG(50:3) enclosing Gckr and Lrpap1. (B) QTL hotspot locus for the 6 lipid species on mouse Chr 19 enclosing Fads 1, 2, 3 and Kat5. The syntenic region on human Chr 11 is shown below. The genomic location of the genes is shown in red in the positive stand for FADS2 and in the negative strand for FADS1, FADS3 and KATS (C and D) 3 QTLs of PC(18:0/16:0) (C) and TAG(51:3) (D) showing epistatic effect by the indicated GWAS identified genes for blood lipids indicated on the left.

FIG. 10 shows plasma and liver levels of 9 TAG species having the most significant correlation between plasma and liver in both diets. Dot plot showing the levels of 9 shortlisted lipids as potential biomarkers of NAFLD in plasma and liver. Lipids in red font indicate pro-NAFLD biomarkers and those in blue font indicate anti-NAFLD biomarkers. P-value <0.05 by Welch's t-test was considered significant between diets.

FIG. 11 shows assessment of TAG NAFLD signatures in mice and humans and their association with biosynthetic pathways. (A-C) C57BL/6J mice: (A) Schematic illustration of the three experimental groups used for validation of the NAFLD signatures; mice on CD (green), mice on HFHS diet from 7-25 weeks (grey), mice starting HFHS diet at 7 weeks and treated with nicotinamide riboside (NR) 9 weeks after the start of HFHS diet till the end of the study (for 9 weeks—therapeutic intervention, magenta). (B) Liver total TAG concentration (normalized to liver weight). (C) Principal component analysis (PCA) of 55 TAG species shows clear separation of only the CD group on PC1. (D-E) Human subjects: (D) PCA of 55 TAG species measured in human plasma from healthy, steatosis and NASH patients. (E) Plasma total TAG levels in human samples. (F-G) BXDs: (F) Correlation matrix showing the Pearson correlation of Atgl mRNA expression in four different metabolic tissues with the pro- and anti-NAFLD TAG signatures in plasma (left) and liver (right) for both diets. (G) Gene ontology biological processes (GO BP) associated with NAFLD TAG signatures. Pathway enrichment analysis was performed with the liver transcripts that significantly correlated (both positively and negatively) with the PC1 of pro- and anti-NAFLD signatures in liver and plasma. Red and blue cells represent the enriched pathways with the positively (scale bar: log 10 p-value) and negatively (scale bar: −log 10 p-value) correlated liver transcripts respectively. For B and E differences in mean TAG levels were compared using two-sample t tests. *p<0.05, **p<0.01, ***p<0.001.

FIG. 12 shows validation of NAFLD TAG signatures in mice and humans. (A) Levels of pro- and anti-NAFLD signatures in C57BL/6J mice fed on CD, high-fat high-sucrose-diet (HFHS) and HFHS diet supplemented with NR, 9 weeks after the initiation of HFHS diet (HFHS+NR). (B) Pearson correlation matrix of the pro- and anti-NAFLD signatures with NAFLD readouts including the NAS score (NAFLD activity score) and liver NAD+ levels. (C) Levels of plasma NAFLD signatures in human subjects with various degrees of NAFLD. (D) Pearson correlation matrix of the NAFLD signatures with NAFLD readouts in human subjects. (E) Pearson correlation of Atgl expression in subcutaneous WAT with pro- and anti-NAFLD plasma TAG signatures in BXD strains. For mice: n=6-9 per group. For human subjects: n=12, healthy; n=7, steatosis; n=14, early stage NASH; n=11, advanced stage NASH. Differences in mean TAG species were compared using two-sample t tests. *p<0.05, **p<0.01, ***p<0.001.

FIG. 13 shows identification of cardiolipin signatures of healthy and fatty liver. (A) Correlation diagram (corrgram) showing diet-independent association of lipid species with liver mass. Lipid species with Spearman's correlation p-value <0.05 with liver mass (both normalized to body weight (%) and unnormalized (weight in g) in both CD and HFD were selected. (B) Spearman correlation network of diet specific significant correlation of lipid species with liver mass in CD (left) and HFD (right). (C) Corrgram of CLs that significantly correlate with liver mass in HFD. (D-F) C57BL6/J mice were fed with CD or high-fat high-sucrose (HFHS) diet for 18 weeks or nicotinamide riboside (NR) supplemented HFHS diet, 9 weeks after the start of the HFHS diet (HFHS+NR). Levels of healthy (D) and unhealthy (E) CL species in livers of the three cohorts. Note that the CLs—CL(LOOPo), CL(LLPoP) and CL(OOOP)—are shown in the figure with an additional CL species because the two are isobaric and inseparable chromatographically. (F) Corrgram showing negative correlation of obesity and NAFLD traits with healthy CL species and positive correlation with unhealthy CL species.

Table 1. Lipid species link to GWAS genes for blood lipid levels and associated metabolic traits. Lipid species having a QTL at the position of the genes identified by GWAS for blood lipids and associated metabolic traits (indicated under GWAS phenotypes).

For each GWAS selected gene, a chromosomal position of ±5 Mb on either side of the gene position was used to match the peak QTL of lipid species. LOD score above 3.5 and local p-value <0.05 was used for the match. Each box with more than one gene represents a syntenic region on the mouse chromosome. The genes in red font (7 out of 10 syntenic regions) are syntenic in humans also. Exclusive literature reference for the GWAS phenotype of the given genes is provided as PMID. Abbreviation: TG: TAG; TC: total cholesterol; HDL-C: HDL cholesterol; LDL-C: LDL cholesterol; CAD: coronary artery disease; BMI: body mass index. An extended version of the table with the metabolic function of the genes and p-value of the QTLs is obtained (data not shown).

Table 2A. List of plasma lipid species measured in each class and their predominant side chain fatty acid (FA) composition. Log2fold change of HFD vs. CD and p-value for each lipid is provided. Nominal p value (Student's t-test) and adjusted P.value (Benjamini-hochberg corrected significance) are indicated for all lipids. The abbreviations of the lipid species are also indicated.

Table 2B. Lipids and their abundance in each class Table 2C. Lipids in each module Table 2D. Heritability of all lipid species Table 2E Spearman correlation between serum and liver lipids.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

The term “and/or” used in a phrase such as “A and/or B” herein is intended to include “A and B”, “A or B”, “A”, and “B”.

The term “at least one” as used herein can refer to one triglyceride (TAG) biomarker, or two TAG biomarkers, or three TAG biomarkers, or four TAG biomarkers, or five TAG biomarkers, or six TAG biomarkers or seven TAG biomarkets selected from the group comprising TAG(52:2), TAG(54:3), TAG(56:3), TAG(50:2), TAG(52:5), TAG(52:4), TAG(54:6). It can also refer to one cardiolipin (CL) biomarker, or two cardiolipin (CL) biomarkers, or three cardiolipin (CL) biomarkers, or four cardiolipin (CL) biomarkers or five cardiolipin (CL) biomarkers selected from the group comprising CL(LLLL), CL(LOOPo), CL(LLPoP), CL(LOOO), CL(OOOP). It can also refer to one monolysocardiolipin (MLCL) biomarker, or two monolysocardiolipin (MLCL) biomarkers, or three monolysocardiolipin (MLCL) biomarkers selected from the group comprising MLCL(LLL), MLCL(LOO), MLCL(LLO).

The term “diagnosing” as used herein refers to assessing whether a subject suffers from the fatty liver disease, or not. The term includes individual diagnosis of fatty liver disease, or its symptoms as well as continuous monitoring of a patient. Monitoring, i.e. diagnosing the presence or absence of fatty liver disease or the symptoms accompanying it at various time points, includes monitoring of subjects known to suffer from fatty liver disease as well as monitoring of subjects known to be at risk of developing fatty liver disease. Furthermore, monitoring can also be used to determine whether a subject is treated successfully or whether at least symptoms of liver disease can be ameliorated over time by a certain therapy. Diagnosing as used herein also refers to diagnosing a predisposition of a fatty liver disease and, thus, predicting whether a subject is at increased risk of developing a fatty liver disease within a predictive window starting from the time when the sample to be analyzed has been taken. Preferably, the predictive window is at least three months, six months, one year, two years, five years, ten years or up to the entire life span of the subject. A subject is at increased risk if the probability by which it will develop the disease is statistically significantly increased with respect to the average or mean probability, i.e. the prevalence for the disease in the respective population from which analyzed subject originates.

The term “fatty liver disease” is well known in the art. Preferably, the term refers to an impairment of the liver. Preferably, said impairment is the result of a surplus of triglycerides, sich as triacylglyceride, that accumulate in the liver and form large and small vacuoles. The symptoms accompanying fatty liver disease are well known from standard text books of medicine such as Stedman's or Pschyrembel. Fatty liver disease may result from alcohol abuse, diabetes mei!itus, nutritional defects and wrong diets, toxicity of drugs or genetic predisposition (see Carulli et al. 2009, Dig Liver Dis. 41(11):823-8. Epub 2009 Apr. 28 “Genetic polymorphisms in nonalcoholic fatty liver disease: interleukin-6-174G/C polymorphism is associated with nonalcoholic steatohepatitis”; or Yoneda et al. 2009, Liver Int. 29(7): 1078-85. Epub 2009 Mar. 3 “Association between angiotensin II type 1 receptor polymorphisms and the occurrence of nonalcoholic fatty liver disease”). Fatty liver disease as used in accordance with the present invention also include the more severe forms thereof and, in particular, steatosis, NASH or NAFDL. Symptoms accompanying these diseases are also well known to the physicians and are described in detail in standard text books of medicine.

As used herein, “treatment” means any manner in which one or more of the symptoms of a disorder are ameliorated or otherwise beneficially altered. Thus, the terms “treating” or “treatment” of a disorder as used herein includes: reverting the disorder (e.g., causing regression of the disorder or its clinical symptoms wholly or partially); preventing the disorder (e.g., causing the clinical symptoms of the disorder not to develop in a subject that can be exposed to or predisposed to the disorder but does not yet experience or display symptoms of the disorder); inhibiting the disorder (e.g., arresting or reducing the development of the disorder or its clinical symptoms); attenuating the disorder (e.g., weakening or reducing the severity or duration of a disorder or its clinical symptoms); or relieving the disorder (e.g., causing regression of the disorder or its clinical symptoms). Further, amelioration of the symptoms of a particular disorder by administration of a particular composition can include any lessening, whether permanent or temporary, lasting or transient that can be attributed to or associated with administration of a composition of the presently disclosed subject matter and/or practice of the presently disclosed methods.

As used herein, the term “subject” relates to animals and, preferably, to mammals. More preferably, the subject is a primate and, most preferably, a human. The subject, preferably, is suffering from a fatty liver disease or is suspected to suffer from a fatty liver disease, i.e. it may already show some or all of the symptoms associated with the disease. Preferably, the subject, however, is besides the aforementioned diseases and disorders apparently healthy. The said subject may also be at increased risk of developing a fatty liver disease, i.e. having a predisposition for a fatty liver disease. Such a subject may be also apparently healthy with respect to a fatty liver disease. A subject being at increased risk may be a subject suffering from diabetes meilitus or an alcohol addict. Moreover, a subject being at increased risk and, thus, having a predisposition for a fatty liver disease, may be a subject which is exposed to toxic substances or harmful drugs or which is affected by a wrong nutritional diet or which has a genetic predisposition.

Lipids are central to all biological processes, from energy storage to forming the matrix of cell membranes to signaling (Han, 2016). They exist in a staggering array of sizes, biophysical properties, and relative abundance. Although structurally lipids are composed of not many ‘building blocks’, they have the potential to generate ˜100,000 different molecular species (http://www.lipidmaps.org/) (Fahy et al., 2005), whose precise function is not easy to decipher given their high complexity. Lipid profiles are determined by the combined influences of genes and environmental factors and their interactions (GxE). The profile changes based on dietary lipids, de novo lipogenesis (DNL), and the hundreds of enzymes that modulate the length and desaturation of fatty acid (FA) chains and their incorporation into more complex lipid molecules. A considerable part of human genome is required to synthesize, metabolize and regulate this lipid diversity. Although it is still a challenge to detect all types of lipid species in a sample, LC-MS-based methods combined with sophisticated software to aid in lipid identification can detect thousands of distinct lipids (Han, 2016; Hyotylainen and Oresic, 2015).

Storage lipids (triacylglycerol, TAG), circulating lipid-protein complexes (lipoprotein particles), and membrane lipids (phospholipids (PL), diacylglycerol (DAG)) as a “class” have previously been linked to the metabolic syndrome, whose features include, obesity, insulin resistance, cardiovascular diseases, non-alcoholic liver disease (NAFLD). (Farese et al., 2012; Han, 2016; Hyotylainen and Oresic, 2014; Puri et al., 2009). However, the precise contribution of most lipid species to the metabolic dysfunction is not well understood. In the past decade, numerous genome wide association studies (GWAS) have identified over 200 genetic variants associated with different lipid class and complex metabolic traits (Diabetes Genetics Initiative of Broad Institute of et al., 2007; Global Lipids Genetics et al., 2013; Surakka et al., 2015; Teslovich et al., 2010). However, with few exceptions (Gieger et al., 2008; IIlig et al., 2010; Shin et al., 2014), these studies focused on the total lipid class and not on individual lipid species, which may be one of the many factors contributing to low variance explained by the loci identified sofar (Johansen et al., 2011). Therefore it is imperative that the focus shifts towards analyzing individual lipid species rather than total class in a population based study. Additionally, human GWAS have rarely identified risk factors that explain more than 5-20% of variance in the target phenotype (Manolio et al., 2009). This problem is particularly compounded by traits, which are strongly modulated by GxE. In contrast, studies on mouse genetic reference populations (GRPs) in which environmental factors can be controlled have been able to provide a stable platform for identifying major genetic, environmental, and GxE factors influencing complex traits, including lipids (Hui et al., 2015; Sittig et al., 2016; Williams and Auwerx, 2015). Studies using only a few hundred such animals have identified loci that explain 30% or more of the phenotypic variance even for complex traits (Burke et al., 2012). While earlier mouse population studies have identified specific loci associated with different lipid class (Hui et al., 2015; Zhang et al., 2012), no study in a GRP has specifically profiled lipid species or identified loci associated with lipid species—nor has GxE been examined for these traits. In this disclosure, targeted lipidomics profiling of 129 lipid species across 271 individuals from 49 distinct inbred strains of the BXD GRP fed either chow diet (CD) or high fat diet (HFD) were performed and that were in parallel subjected to an extensive battery of metabolic tests (Williams et al., 2016). Fasting lipid species in plasma from different lipid class including free fatty acids (FFA), TAG, DAG, phosphotidylcholine (PC), phosphotidylethanolamine (PE), phosphatidylinositol (PI), phosphotidylglycerol (PG) and conenzymeQ (CoQ) were measured. Both genetic (quantitative trait locus (QTL) mapping) and multilayered omics (correlation, network analysis, module trait correlation) approaches were used to gain a comprehensive understanding of the genetic and dietary impact on lipid species and to uncover the potential of lipid species to predict metabolic health. Over 200 QTLs associated with 70% of the lipids measured were identified. 91 of these QTLs, in 28 different loci harbored 35 genes that were associated with lipid levels and metabolic syndrome associated traits in human GWAS. Lastly, the multi-scalar approach enabled to link a subset of TAG species as biomarkers of non-alcoholic fatty liver disease (NAFLD) that was validated is a mouse model of NAFLD and in human NAFLD subjects.

Plasma Lipidomics Profile Across the BXD GRP Shows a Coherent Dietary Effect

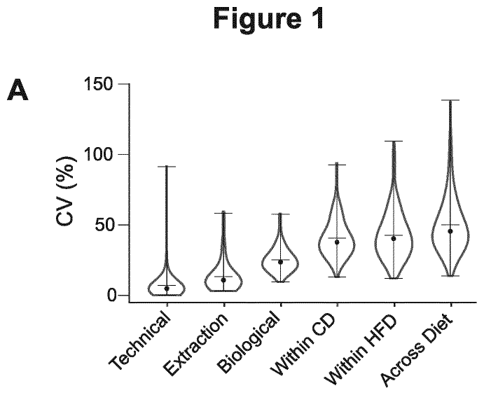

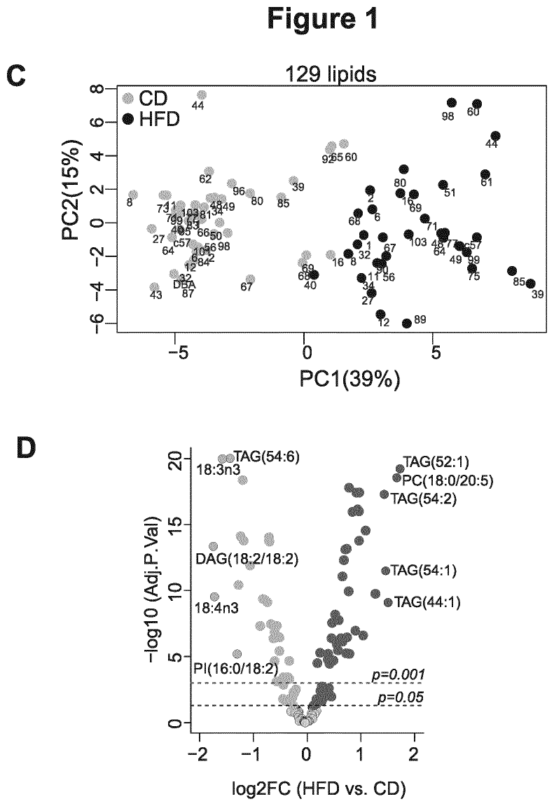

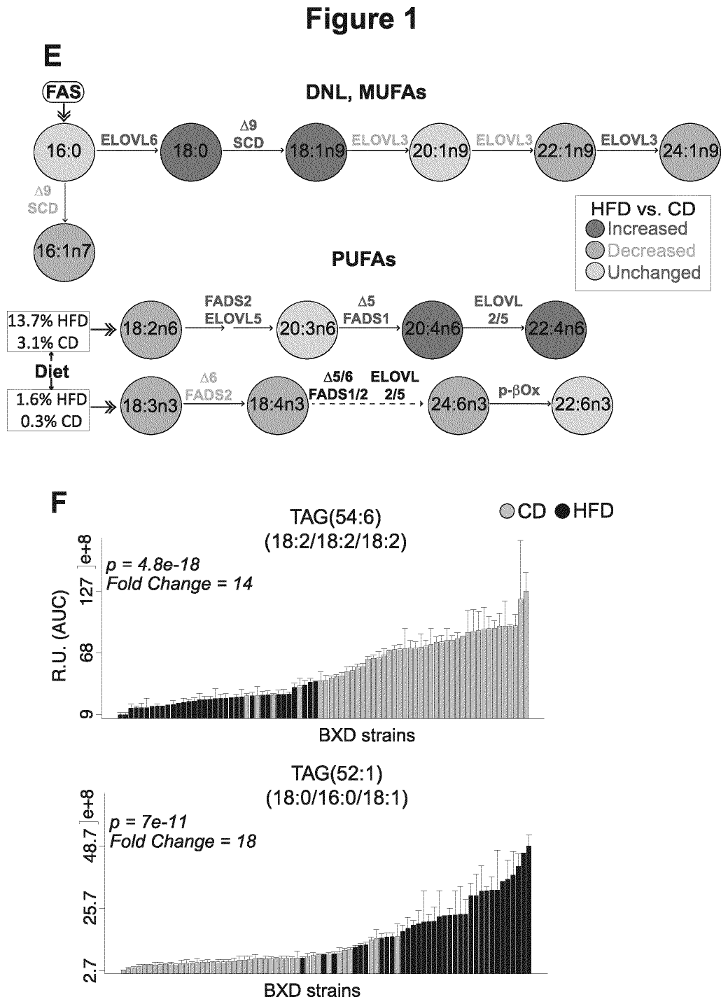

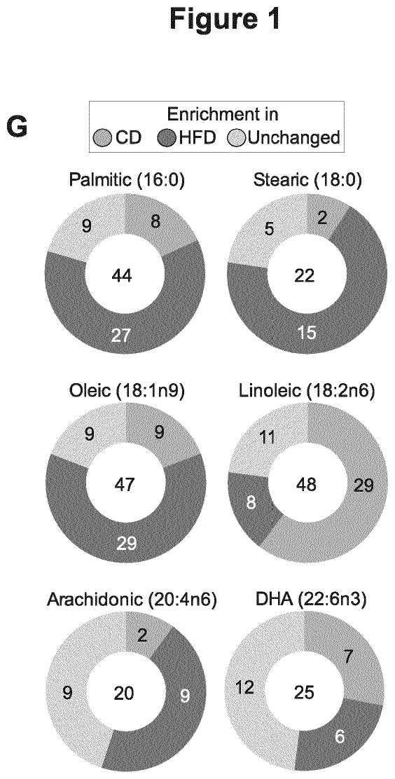

In an attempt to characterize the plasma lipidome signatures, a targeted lipidomics approach was used to measure 129 lipid species from different lipid classes in 78 BXD cohorts, 44 cohorts fed CD and 34 fed HFD. While on their respective diet, mice underwent extensive metabolic phenotyping (Williams et al., 2016; Wu et al., 2014). At the end of the phenotyping program, over-night fasted mice were sacrificed and plasma samples were used for lipidomics analyses. The detection of lipid species was based on their abundance, stability, polarity and ease of ionization. Lipid species characterized by their number of carbon atoms and double bonds in constituent acyl residues were measured, identifying 8 different lipid class including FFAs (16 species), TAG (53 species), DAG (6 species), PC (28 species), PE (15 species), PI (7), PG (2) and CoQ (2) (Table 2A). The quality of the mass spectrometry (MS) measurement was assessed by its coefficient of variation (CV) for technical and extraction replicates, which was ˜5% and ˜10% of the overall variation respectively, suggesting a very good reproducibility (FIG. 1A). The variation within biological replicates (˜25%) was much lower than variation across strains in either diet and across diet (˜45%, FIG. 1A).

To identify lipid species and strains that show similar characteristics, hierarchical cluster analysis was used, which demonstrated that the BXD cohorts were clearly—but notably, not 100% segregated, indicating that genetic background can overpower even the massive effects of CD vs. HFD on lipid profiles (FIG. 1B). Notably, the four CD cohorts that cluster most closely with the HFD cohorts—BXD92, 65, 60, and 39—are among the most obese and having the largest livers of the CD-fed cohorts (data not shown). Conversely, the last 5 BXD strains (BXD 27, 12, 89, 40, 34) on HFD that cluster close to CD cohorts are also among the 20% of the BXD strains on HFD that have low body and liver weight (data not shown). To investigate the potential lipidome differences between the CD and HFD cohorts by a different method, principal component analysis (PCA) (FIG. 1C) was performed. The first dimension (PC1) clearly segregated the BXDs by diet, confirming the findings obtained by the hierarchical cluster analysis (FIG. 1C). However, the variance explained by the sum of the first two principal components is only 54%, which indicates that the lipidomic profile of the BXDs is highly variable in both diets (FIG. 1C). Of the 129 lipid species measured, 94 were significantly changed across the two cohorts (51 upregulated in HFD vs. CD and 43 downregulated in HFD vs. CD), (FIG. 1D and Table 2A), suggesting a clear diet effect on majority of the lipids. The top 10 most significantly different lipid species between the two groups were also the most variable lipids influencing the principle component PC1 (FIG. 1D, lipids named on the graph).

It was next evaluated the impact of diet on DNL, monounsaturated fatty acid (MUFA) (FIG. 1E, top) and polyunsaturated fatty acids (PUFA) (FIG. 1E, bottom) synthesis—major determinants of the lipid profile—by analyzing the levels of FFAs in both diets. Stearic (18:0) and oleic (18:1n9) acids were significantly increased in HFD cohorts, indicative of increased DNL under HFD (FIG. 1E, top). The desaturation and elongation products of 18:1n9 were, however, not increased in HFD cohorts indicating an accumulation/enrichment of oleic acid side chain in lipid species increased with HFD (FIG. 1E, top, FIG. 1G). In contrast to DNL and MUFA synthesis, no clear dietary difference was observed in PUFA synthesis (FIG. 1E, bottom). Though the essential fatty acids (EFAs) including, linoleic (18:2n6) and linolenic (18:3n3) acids were 4.4 and 5.3 times higher in the diet of HFD cohorts, their levels were not higher in the plasma of HFD mice (FIG. 1E, bottom). This reflects increased utilization of the EFAs as an energy source in the HFD cohorts, as evidenced by increased levels of 20:4n6, 22:4n6 and peroxisomal-β oxidation (FIG. 1E, bottom).

It was next verified the potential of plasma FFAs as representative of DNL and MUFA synthesis by analyzing the enrichment (HFD vs. CD) of the esterified forms of these FFAs in other lipid species. For the majority of the lipid species, their increase or decrease in either diet (Table 2A) could be easily predicted by their FA side chain composition. For instance, if the side chain was enriched in a FA that was increased in CD then that lipid species was increased in CD as illustrated for two lipid species increased in either CD or HFD (FIG. 1F). TAG(54:6), predominantly comprising 18:2n6, is significantly increased in CD cohorts (FIGS. 1F and 1E, bottom); whereas, TAG(52:1), predominantly comprising 18:0, 16:0 and 18:1, is significantly increased in HFD cohorts (FIGS. 1F and 1E, top).

In order to evaluate the effect of diet on relative enrichment of side chains in lipid species, it was next calculated the side chain composition of all lipid species measured. The majority of side chains comprised of palmitic (16:0), stearic (18:0), oleic (18:1n9), linoleic (18:2n6), arachidonic (20:4n6) and docosahexaenoic acid (DHA; 22:6n3). Lipid species with at least one palmitic, stearic and oleic acids in their side chain were increased in HFD cohorts, while lipid species with at least one linoleic acid in their side chain was increased in CD cohorts. Lipid species with an arachidonic acid side chain were either increased in HFD or unchanged and those with a DHA side chain were unchanged (FIG. 1G). The dietary enrichment of stearic, oleic, linoleic and arachidonic acids and DHA seen here also reflects the FFA profile seen in FIG. 1E. Taken together, these results suggest that diet has a strong influence in determining the lipid profile and in HFD there is a switch towards greater utilization of EFAs to meet the energy demand (metabolic flexibility). Additionally, results disclosed herein also suggest that FFAs in general can reflect the systemic profile of other lipid species.

Unweighted Correlation Networks of Lipid Species and Identification of Predictors of Metabolic Health

Next, unweighted correlation network analysis of all the lipid species was performed, to obtain an overview of the interaction between the different lipid species and lipid class (FIGS. 2A and 2B). In both CD and HFD cohorts, lipid species were highly correlated both within and across different lipid class. Positive edges/correlations are ˜2.2 times more prevalent than negative edges in both the diets. Both the CD and HFD network showed a strong dense grouping of TAGs, PCs and DAGs (FIGS. 2A and 2B, red ellipse). This cluster is indicative of the PC and TAG synthesis from DAG intermediates (Han, 2016). TAGs with high carbon number (56-64) are not connected in the CD network but are integrated in the HFD network (FIGS. 2A and 2B, green ellipse). Coenzyme Q9 shows strong negative correlation with TAG species and palmitic acid in CD, but not in HFD. The dense subnetwork of different lipids indicates that lipid species across different class are highly co-regulated and interconnected, and that a change in one or more species would impact on the levels of many other lipid species.

Based on this notion, it was considered that it may be possible to predict a metabolic phenotype of mice based on their lipid species profile in plasma and vice-versa. From the extensive phenotypic profiling performed in these mice (Williams et al., 2016), 31 unique traits as predictors of metabolic health/fitness were shortlisted. These traits covered a wide spectrum of tests including, fat and lean mass, physical fitness (treadmill exercise, activity wheel, VO₂ max), oral glucose tolerance test, heart rate and plasma biochemical markers (ALT, AST, cholesterol etc), among others. Spearman correlation was performed for all lipid species and the shortlisted traits (data not shown). Correlation rho value was used to perform a heatmap analysis with unsupervised hierarchical clustering of all lipid species (FIG. 6A). On both CD and HFD, two lateral clusters of lipid species could easily be identified, which correlated with most of the metabolic traits (FIG. 6A). From these two clusters in each diet (FIG. 6B), 38 plasma lipid species common to both diets were identified, which showed the strongest correlations with metabolic traits (FIGS. 2C and 2D). Amongst them, 21 lipids were identified as “healthy markers” in both diets (FIGS. 2B and 2C, vertical green cluster), since they showed positive correlations with 9 healthy metabolic traits (FIGS. 2B and 2C, horizontal green cluster) and negative correlations with 20 unhealthy traits (FIGS. 2B and 2C, horizontal red cluster). While 17 lipids were identified as “unhealthy markers” (FIGS. 2B and 2C, vertical red cluster), as they negatively correlated with healthy traits (FIGS. 2B and 2C, horizontal green cluster) and positively correlated with unhealthy traits (FIGS. 2B and 2C, horizontal red cluster).

Note that the trait—FFA, clusters with healthy metabolic phenotypes, including heart rate (indicated in grey on the right side of FIGS. 2C and D). The fact that FFAs provide 60-70% of the heart's energy requirement (van der Vusse et al., 2000) and that the mice were fasted before sacrifice may explain the clustering of FFAs with healthy phenotypes. Therefore FFAs may be considered as a false positive metabolic health predictor. Additionally, HDL correlated positively with healthy lipids and negatively with unhealthy lipids in HFD, whereas in CD it did not have significant correlation with any lipids, except for PC(38:2). This suggests that HDL is a good predictor of healthy traits under HFD only. Lipids highlighted in blue font (21 in CD and 12 in HFD), could be considered as the top best predictors of metabolic fitness because they show significant correlation with more than 50% of the metabolic traits in either diet. Interestingly, only 7 of the 38 lipids identified as metabolic health predictors are amongst the most abundant lipids in their class [PC(20:4/18:0), PC(20:4/16:0), PC(22:6/16:0), PC(36:1), TAG(52:2), TAG(52:4), TAG(54:3)]; the remaining 31 lipids have low abundance in plasma (Table 2B). With the exception of TAG(52:4), 6 out of 7 abundant species are negative markers of metabolic health. Taken together, these data suggest that, lipid species can be used as powerful predictors of metabolic health and that the most abundant species may not necessarily be the good predictors of health.

Weighted Correlation Network Analysis (WGCNA) Reflects Strong Dietary Impact and Identifies Lipid Modules as Predictors of Metabolic Health

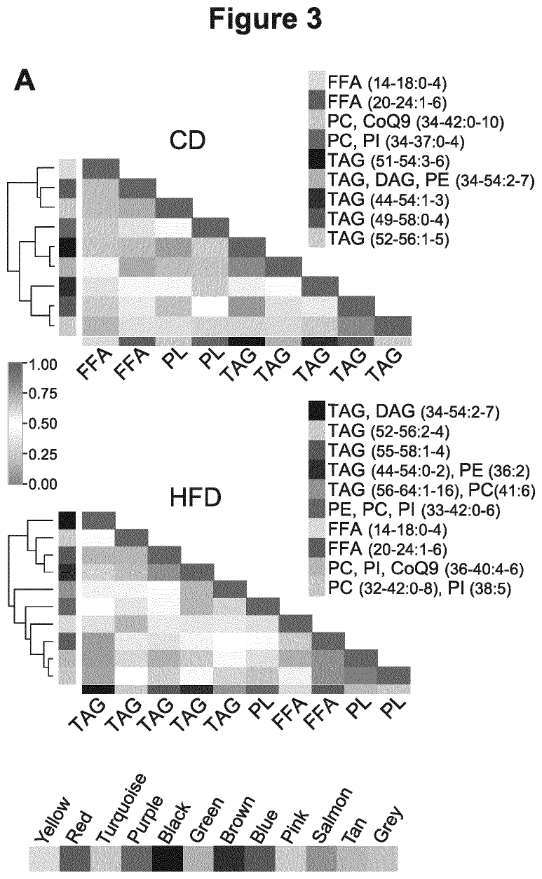

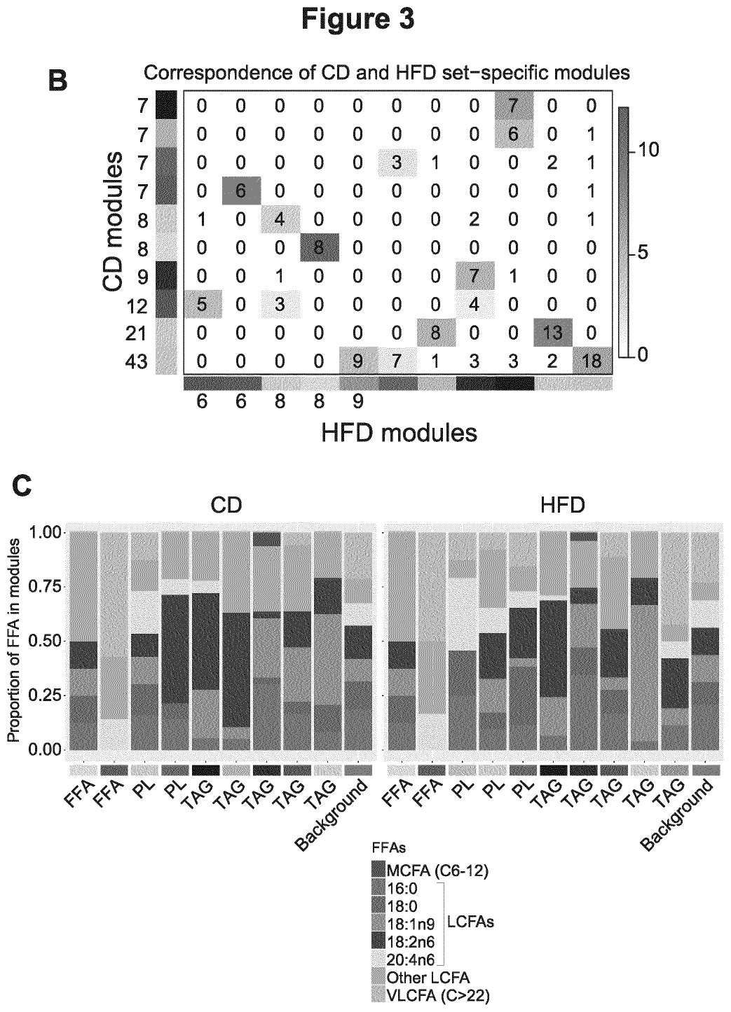

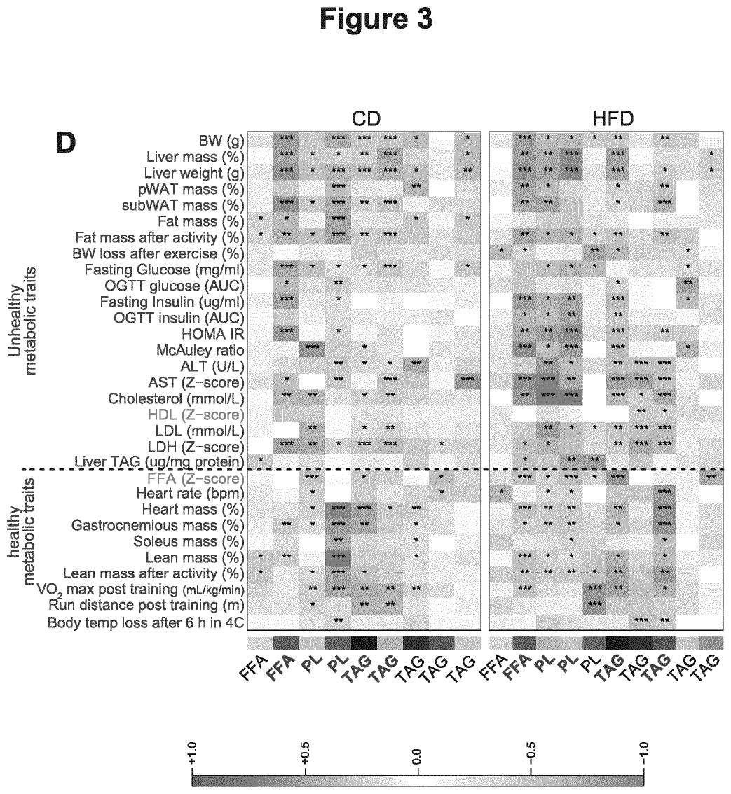

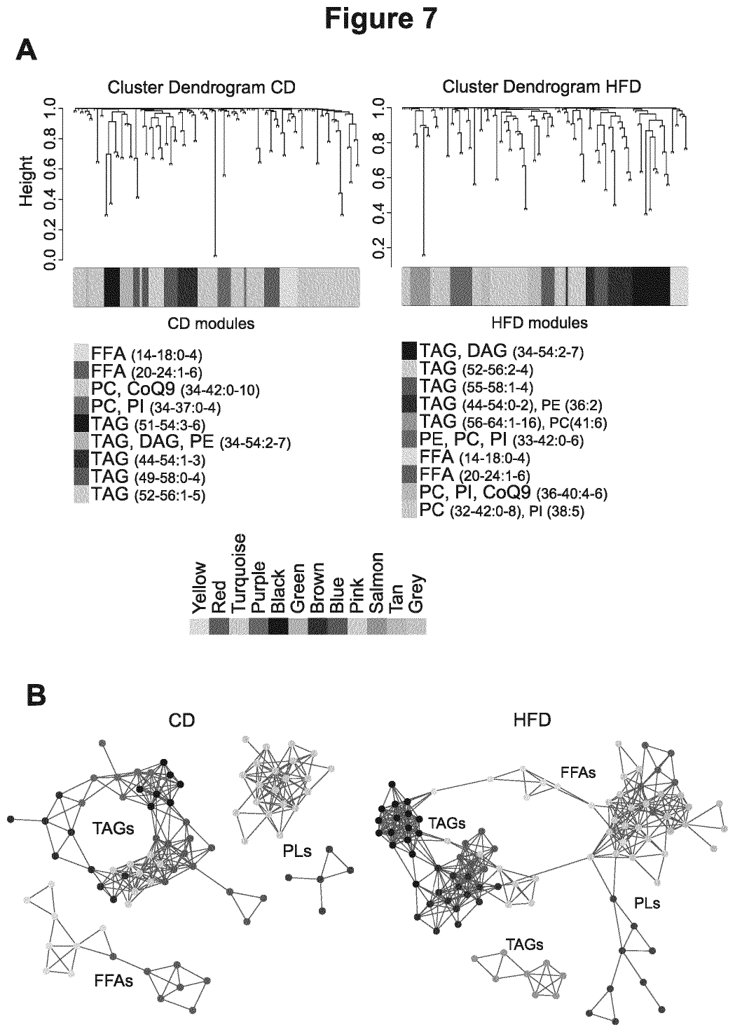

In order to reduce the complexity of the plasma lipid profile, WGCNA was performed (Langfelder and Horvath, 2008) in both CD and HFD cohorts. WGCNA helped identify clusters of highly correlated lipids, which are potentially co-regulated within 9 and 10 different modules in CD and HFD, respectively (Table 2C). Each module is named and represented by different colors according to the major lipid class contained in them (FIG. 3A, S2 and Table 2C). Modules were dominated by lipids in the same class identifying 5 TAG, 2 FFA, and 2 PL modules in CD, and 5 TAG, 2 FFA and 3 PL modules in HFD; lipids not clustered in any module were assigned to the grey color and considered as background (FIG. 3A, S2 and Table 2C). In general modules containing lipids of the same class showed high adjacency (FIG. 3A) as well as correlation between them (FIG. 7B). In addition to the lipid class, each module also grouped lipids with similar carbon chain length and degree of unsaturation (FIG. 3A, and Table 2C), demonstrating a good agreement between the WCGNA and the biological significance.

It was next compared the impact of diet on the lipid composition of each module. Correspondence analysis between CD and HFD evidenced a general moderate-low conservation in the lipid modules, indicating a strong diet effect on module formation (FIG. 3B). Among all modules, only the FFA (red and yellow) modules were conserved between CD and HFD, suggesting the diet-independent nature of these clusters (FIG. 3B). Since mice were fasted, conservation of FFA modules could indicate that the mobilization of FFA upon fasting was similar in both diets. TAG and PL modules showed a low or null conservation between the diets, indicating a strong dietary impact on the module composition (FIG. 3B). For example, the black and green TAG modules in CD were fused in HFD as a single module (black). Conversely, PLs in the turquoise module in CD corresponds to turquoise and tan modules in HFD, indicating that in HFD these PLs show differential clustering than in CD (FIG. 3B and Table 2C). Interestingly, the salmon-colored module, which only appeared in HFD and did not cluster with any other module, is composed of TAGs with high number of carbons (56-64) in their side chains, indicating a specific effect of the diet over these TAGs (FIGS. 3A and 3B), and consistent with the unweighted network analysis (FIG. 2A-B).

It was then analyzed the FA side-chain composition of the lipids in each module, in order to have a global overview of the degree of unsaturation in each module. The side chain FA profile varied between modules in each diet, however, in general the proportion of FAs in each module showed a high conservation between CD and HFD (FIG. 3C), contrary to the individual lipid composition (FIG. 3B). For example, medium-chain fatty acids (MCFAs; C10 and C12) appear only in TAGs of the brown module in both diets, and proportions of palmitic acid (16:0) were conserved across the CD and HFD modules (FIG. 3D; red and orange bars respectively). Notably, the grey module (background lipids) showed the same mixed FA composition in both diets, not being enriched in any particular FA (FIG. 3C). In line with FIG. 1G, long-chain fatty acids (LCFAs; C13-21) were among the most abundant side chain FAs in both diets (FIG. 3C). When comparing the PL modules in the two diets, arachidonic acid (20:4n6) and very long-chain fatty acids (VLCFAs) were relatively enriched in HFD while linoleic acid (18:2n6) was enriched in CD (FIG. 3C; yellow, brown and purple bars respectively). Modules corresponding to FFAs (red and yellow) showed quite different composition and were consistent in both diets; the yellow module was composed by LCFAs while the red module was predominantly composed of VLCFAs (FIG. 3D). Interestingly, among the TAG modules in HFD, the salmon module (which appears only in HFD), showed the highest proportions of arachidonic acid and VLCFAs and therefore the lowest proportions of LCFAs (FIG. 3C). These results firmly indicate that although the diet has a strong impact on lipid clustering/modules (FIGS. 3A and 3B), the side-chain composition of the modules across diet (FIG. 3C) seems predominantly genetically regulated and driven by their biophysical property.

To assess the functional relevance of the lipid modules identified, it was performed a correlation analysis between the modules and a set of metabolic traits used in the previous figure (FIGS. 2C and 2D). Nine metabolic traits were chosen as predictors of good health (FIG. 3D; green font) and 21 traits as predictors of unhealthy metabolism and or the metabolic syndrome (FIG. 3D; red font). 5 modules (3 in CD and 2 in HFD) were identified as the best predictors of healthy metabolism (FIG. 3D; green font) and another 5 (2 in CD and 3 in HFD) as the best predictors of unhealthy metabolism (FIG. 3D; red font). The 5 best predictors of metabolic fitness correlated positively with healthy traits and negatively with unhealthy traits (FIG. 3D, modules indicated in green font). Interestingly, all these 5 modules were enriched in linoleic acid side chain (FIG. 3C). In contrast, the 5 modules identified as the predictors of unhealthy metabolic traits correlated negatively with healthy traits and positively with unhealthy traits (FIG. 3D, modules indicated in red font). The FA enrichment was quite variable across these 5 unhealthy modules. (FIG. 3C). Interestingly, the pink TAG module was enriched in the abundant FAs namely, oleic acid and SFAs showed significant positive correlation with obesity phenotypes including body weight, fat mass, liver mass and fasting glucose. Of note, the liver triglyceride concentration showed the strongest positive correlation with the 2 PL modules in HFD (FIG. 3D, turquoise and purple module) and no significant correlation with TAG modules, suggesting that the plasma levels of some of these PLs could be a good indicator of liver steatosis, as already reported (Anjani et al., 2015; Li et al., 2006; Ma et al., 2016). Of importance, the module-metabolic trait correlation was in line with the individual lipid to metabolic trait correlation (FIGS. 2C and 2D) suggesting that sets of lipid modules can also be a powerful predictor of metabolic fitness.

Plasma Lipid Species are Highly Heritable in Both Diets and Influenced by Many Genomic Loci

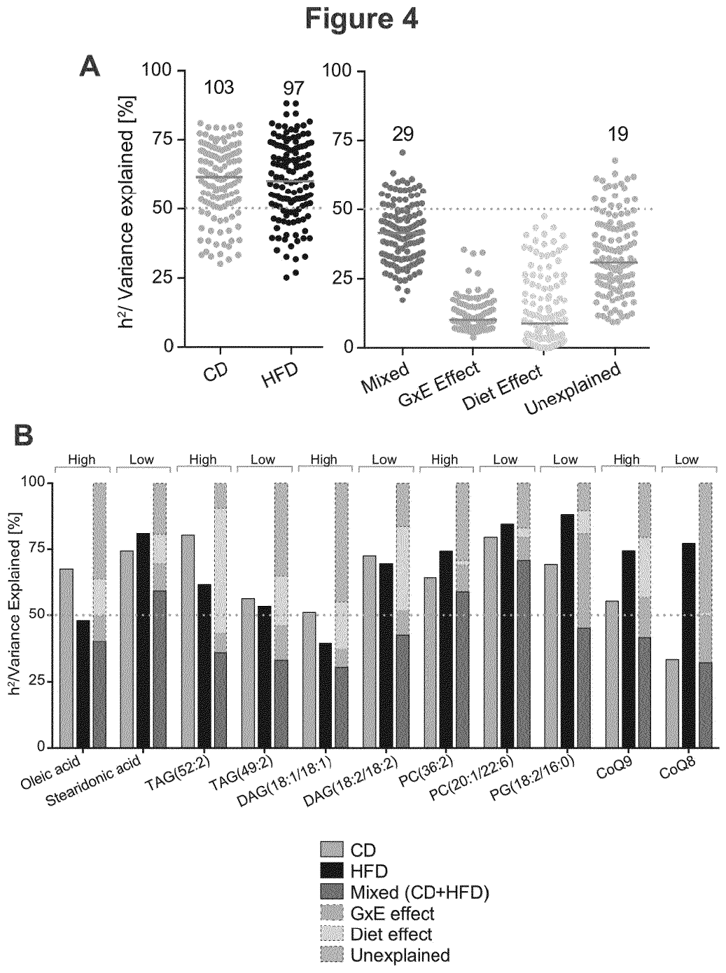

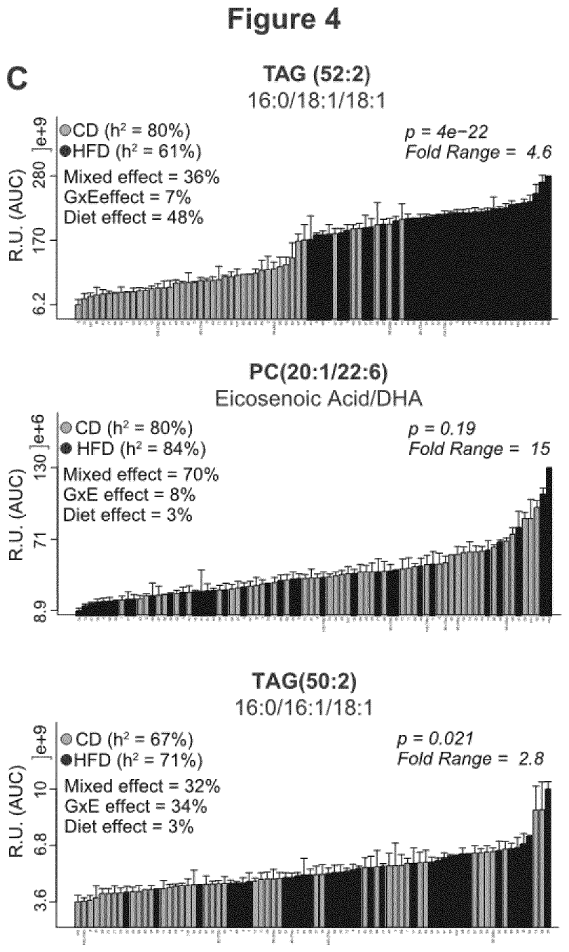

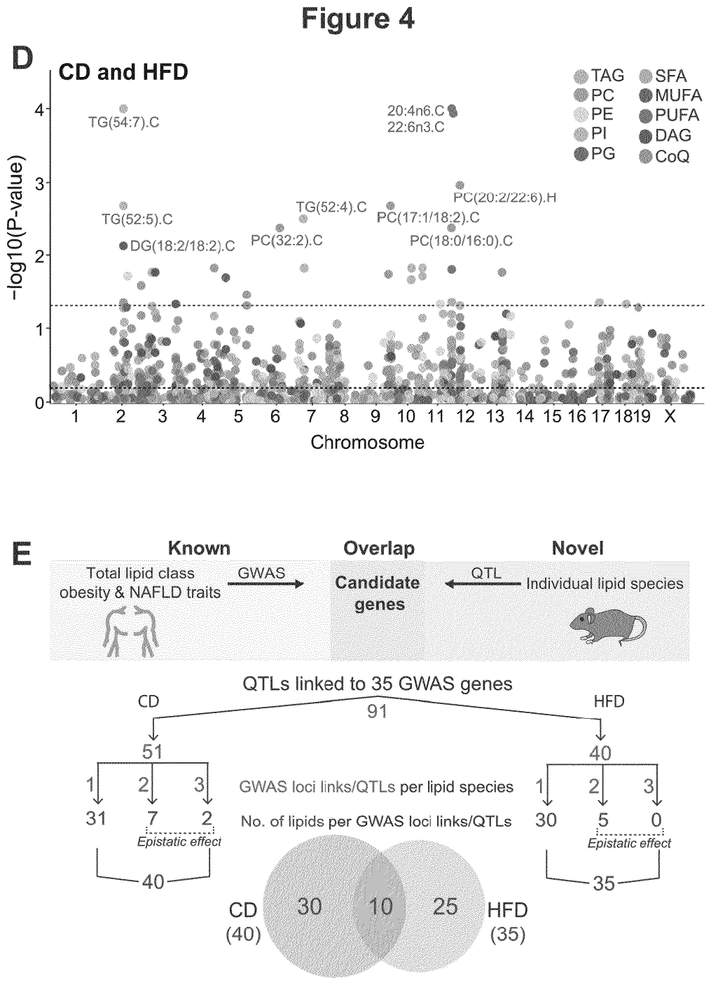

Next, it was assessed the degree of genetic, dietary and GxE regulation of lipid species. Heritability (h²; percentage of trait variation attributed to additive genetic factors) was calculated for all lipid species within dietary groups (CD and HFD) and across all cohorts combined (CD+HFD/Mixed) (FIG. 4A and Table 2D). Additionally, the variation explained by GxE, diet and non-genetic, non-dietary variance (Unexplained) was also calculated (FIG. 4A and Table 2D). Within a dietary cohort, more than half of the observed variance in lipid levels could be explained by genetic differences across strains (i.e. h² z 50%) for the strong majority of lipid species (80% in CD and 75% in HFD). Conversely, when dietary cohorts were combined, only 22% of lipids had h² above 50%. However, even when the dietary cohorts were mixed, the diet-independent genetic factor (FIG. 4A, “Mixed”) was the strongest contributor to variance explained. The contribution of GxE (“GxE Effect”) or diet alone (“Diet Effect”) did not explain even 50% of observed variance for a single lipid species. Together, these three controlled factors—genetics, environment, and GxE—explained more than half of the variance for 85% of all lipid species (FIG. 4A, “Unexplained”). These results indicate a high degree of genetic regulation of lipid species.

It was then examined the h² of the most and least abundant lipid species in each lipid class (FIG. 4B). The h² explained by genetics for a single lipid species varied substantially between cohorts (FIG. 4B; the heights of the green, black, and red+blue bars). For TAGs, the most abundant species (TAG(52:2)) had relatively high h² in both diets when compared to the least abundant species in this class (TAG (49:2)). For FFAs and DAGs, the trend was opposite: the least abundant species had high h² in either diet (Figure. 4B). For PLs, the h² was high in both low (PC(20:1/22:6), PG(18:2/16:0)) and high (PC(36:2)) abundant species in both diets (Figure. 4B). Certain lipids had extremely high h² values in both dietary groups (e.g. stearidonic acid, PC(36:2), PC(20:1/22:6), indicating a high degree of genetic regulation completely independent of diet (FIG. 4B; red bars). As such, for these lipids the effects of diet and GxE were low (Figure. 4B; yellow and blue bars respectively). Lipids like TAG(52:2) had high dietary variance (Figure. 4B; yellow bar). Finally, some lipids (e.g. DAG(18:1/18:1) and CoQ8) had high variance which could not be explained by genetic, dietary or GxE factors (Figure. 4B; brown bars). Taken together, these data indicate that the majority of variance in lipid species levels can be explained by the genetic and environmental factors, it was controlled in this disclosure (i.e. the height of the red+blue+yellow bar, FIG. 4B). Though the diet effect on all lipid species was relatively low (<50%) (FIG. 4A), these data suggest that diet can change the genetic factors regulating the lipids.

For most lipids, the difference in their levels between the diets reflected their variance explained by genetics (CD+HFD/Mixed), diet and GxE (FIG. 4C). For instance, TAG(52:2) levels were significantly increased by HFD, where the diet explained its maximal variance (48%). Conversely, PC(20:1/22:6) levels were not different between the diets, showing high h² in both diets and mixed variance explained its maximal variance (70%) thus reflecting a primary genetic regulation. However, for TAG(50:2), though its levels were significantly increased in HFD, the fold range was low (1.16 fold) and the variance explained by diet was also low (3%), whereas the variance explained by mixed effect and GxE was similar (32% and 34% respectively), reflecting a similar genetic and GxE regulation (FIG. 4C).

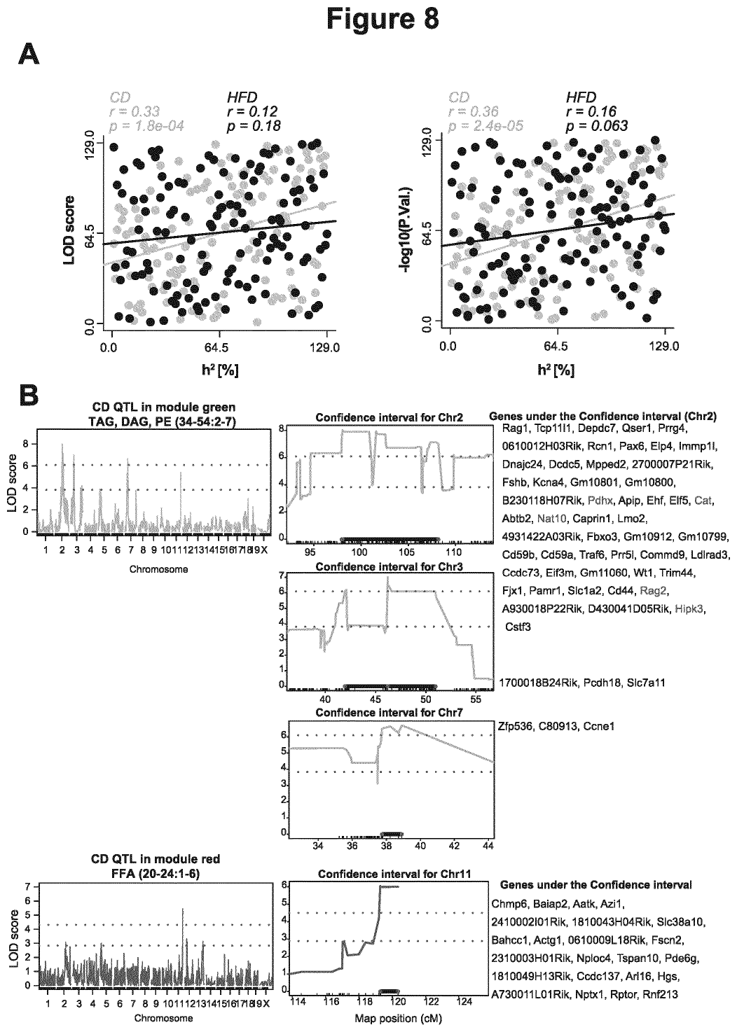

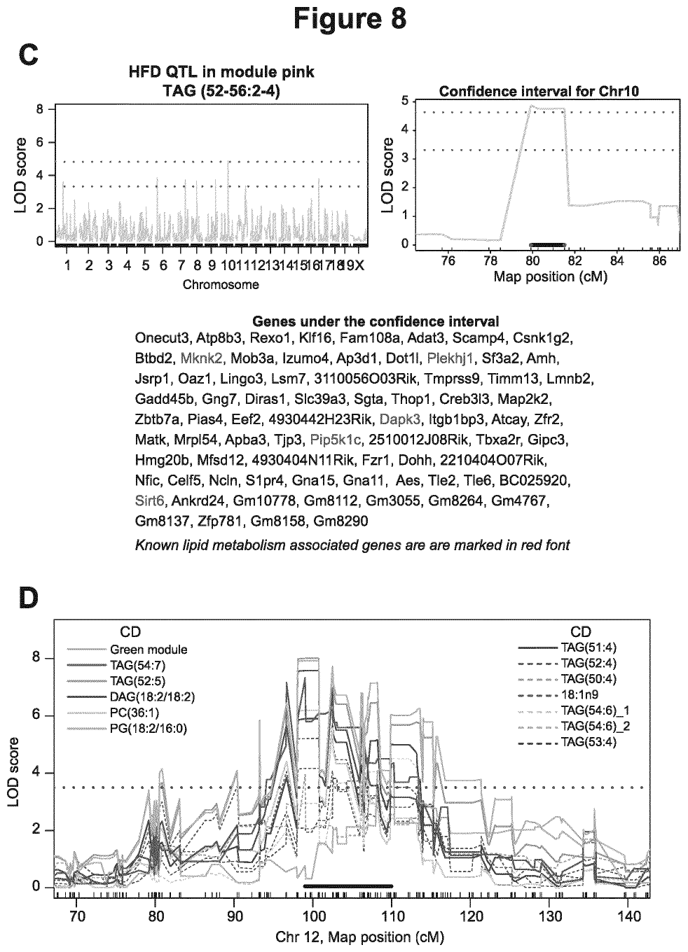

Next, quantitative trait loci (QTL) were mapped for all lipid species and lipid modules (data not shown). The most significant QTLs—those with the highest LOD score—correlated positively with lipids' h² and their significance (FIG. 8A). In general, there were more QTLs in CD compared to HFD (data not shown), suggesting that the environmental effect of HFD could outweigh or modulate the genetic regulation of some lipids. In CD, it was detected 23 lipid QTLs with genome-wide p-value <0.05 and 157 QTLs with local p-value <0.05 and LOD >3.5. In HFD, 11 lipid QTLs were detected with genome-wide p-value <0.05 and 129 QTLs with local p-value <0.05 and LOD >3.5 (data not shown)(FIG. 4D). Additionally, in CD, 4 lipid module QTLs were detected (Consortium, 2015) (modQTLs) at genome-wide significance and 15 modQTLs with local significance and LOD threshold, whereas in HFD, 1 and 20 were detected at the same respective thresholds (data not shown). It is not surprising that most lipids had more than 1 significant or suggestive QTLs, given the fact that many independent processes and pathways regulate lipid species. Only one QTL position, for DAG(18:1/18:1) at chr 3 @ 32.2-33.7 Mb was consistent between CD and HFD, which reiterates that diet can change the genetic factors regulating the lipids species (data not shown). A prominent metabolic hotspot region on chr 2 between 99-110 Mb in CD was observed. Six lipid species with genome-wide significance and additional six lipids with local significance mapped to this region (FIG. 4D, S3B). This QTL region also coincided with the green modQTL comprising seven lipids, of which five lipids (TAG(54:7), TAG(52:5), DAG(18:2/18:2), TAG(52:4), and TAG(50:4)) were common with the individual lipid species QTL at this region (FIG. 8D). This region is enriched in genes involved in FoxO signaling, lipid transport, TAG and DAG metabolic process and phospholipid acyltransferase activity (FIG. 8D).

Lipid Species Link to GWAS Genes for Blood Lipid Levels and Associated Metabolic Traits in Humans

Human GWAS studies have identified many genetic variants associated with the total concentration of different lipid class (Diabetes Genetics Initiative of Broad Institute of et al., 2007; Global Lipids Genetics et al., 2013; Kathiresan et al., 2009). Taking advantage of the novel lipid species QTLs herein disclosed (data not shown) and the known human GWAS loci for the total lipid class and their associated metabolic traits, it was attempted to extrapolate between the two. Of the many lipid QTLs herein disclosed, several of them (51 QTLs in CD and 40 in HFD) linked to 35 out of ˜300 genes pre-selected from GWAS for metabolic traits including plasma TAG, total cholesterol (TC), HDL cholesterol (HDL-C), LDL cholesterol (LDL-C), coronary artery disease (CAD), glucose and insulin levels, obesity and NAFLD traits (FIG. 4E, Table 1). Importantly, 7 syntenic GWAS regions were found in humans (0.1-23 Mb) that were also syntenic in mice (0.3-7 Mb), in addition to 3 others, which were syntenic only in mice. (FIG. 4E, Table 1, genes in red font). These 51 CD and 40 HFD QTL link to GWAS genes were from 40 lipids in CD and 35 in HFD, having 10 lipids (but not the QTL position) in common. 31 CD and 30 HFD lipids mapped to QTLs containing a single known GWAS gene; whereas, 9 CD and 5 HFD lipids were linked to 2-3 GWAS genes (different loci), suggesting an epistatic regulation of these lipids by different genes (FIG. 4E). Additionally, the majority of the GWAS genes, were linked to 3-16 lipid species (Table 1). Given the dense correlation of lipids in the network (FIGS. 2A and 2B) and modules (FIGS. 3A and 3B), it is not surprising to find the widespread genetic influences on plasma lipidome.

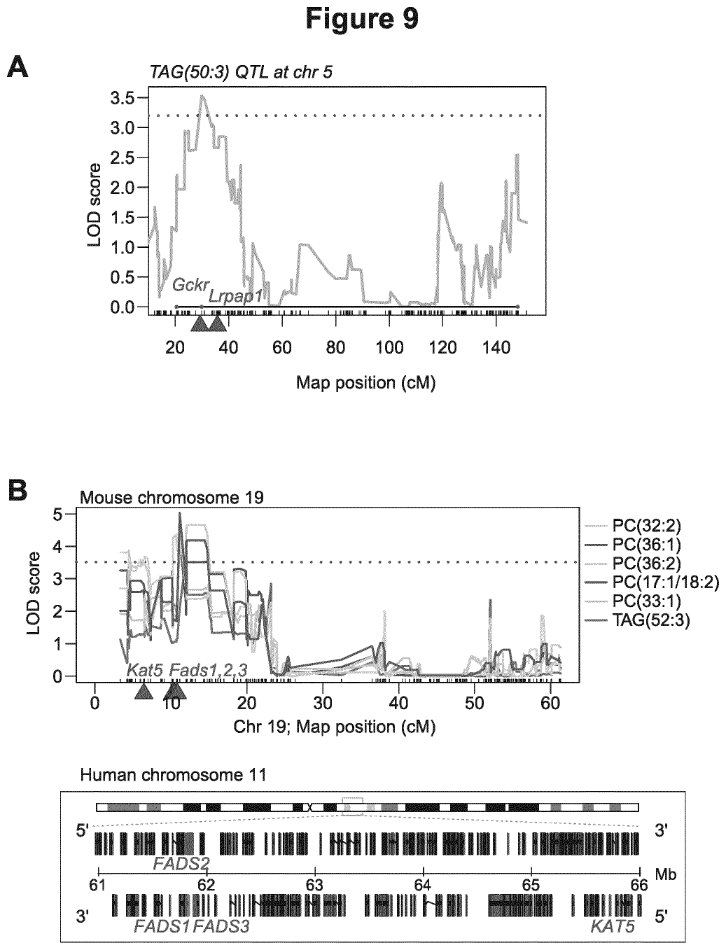

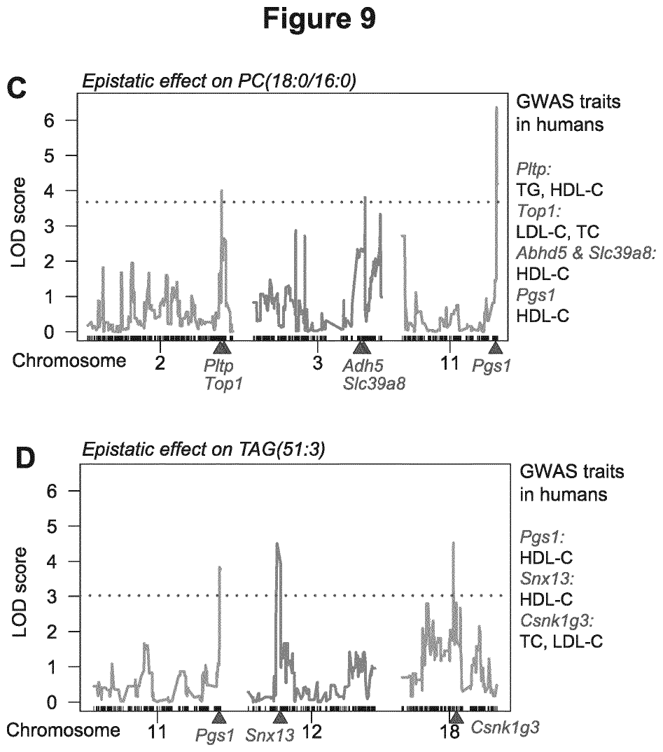

The Gckr locus, encoding the glucokinase (GCK) regulatory protein (Gckr), is one of the most robust loci implicated in TAG metabolism (Diabetes Genetics Initiative of Broad Institute of et al., 2007). It has been replicated in GWAS of plasma TAG concentration, NAFLD and hypertriglyceridemia by an excess of rare variants in patients(Johansen et al., 2011; Kathiresan et al., 2009; Speliotes et al., 2011). Of all the TAGs measured, TAG(50:3) having side chain FA composition of (16:1/16:1/18:1) had a QTL at the Gckr locus. Lrpap1 (LDL Receptor Related Protein Associated Protein 1), at the same locus as Gckr has been associated with TAG, TC and LDL-C(Table 1, FIG. 9A). Fatty acid desaturase (Fads 1, 2 and 3) is another gene which has been associated with TAG, PLs and type 2 diabetes in numerous GWAS studies (Diabetes Genetics Initiative of Broad Institute of et al., 2007; Johansen et al., 2011; Kathiresan et al., 2009; Speliotes et al., 2011). In line, one TAG and 5 PC species had a QTL at the Fads locus. Lysine acyltransferase (Kat5) associated with HDL-C levels, shares the same locus as Fads. Interestingly this locus (with Fads and Kat5) is syntenic in mice and humans (Table 1, FIG. 9B). PC(18:0/16:0) and TAG (51:3), were the two lipid species showing epistatic regulation by 3 different loci on different chromosomes. For PC(18:0/16:0), the 3 loci (2 of which are syntenic with human loci) have been associated with TAG, TC, LDL-C and HDL-C, while for TAG (51:3), all the 3 loci has been associated with TC, LDL-C and HDL-C(Table 1, FIG. 9C, S4D). Taken together, these data suggest that a large number of GWAS genes linked to total lipid levels could in fact be regulating individual lipid species.

Identification of Lipid Species as Biomarkers of Non-Alcoholic Fatty Liver Disease (NAFLD)

Plasma TAG concentration is a complex polygenic trait that follows a rightward-skewed distribution in the population, due to the wide range hypertriglyceridemias encountered (Johansen et al., 2011). Even the hepatic TAG content varies widely among individuals: from <1% to >50% of liver weight (Browning et al., 2004). Recent GWAS have identified both known and novel loci associated with plasma and liver TAG concentration. However, genetic variation at these loci explains only 10% of the overall TAG variation within the population corresponding to 25-30% of the total genetic contribution to TAG variability (Johansen et al., 2011; Speliotes et al., 2011). This may be partly due to the fact that total TAG concentration is always considered as a proxy for all TAG species. However, it has been shown that all TAGs do not follow similar trend in NALFD (Gorden et al., 2015; Hyysalo et al., 2014; Oresic et al., 2013; Puri et al., 2009). Measurement of TAG species is hence imperative since different species exhibit contradictory effects and have vastly different functions and biological impact (FIGS. 2A and 2B and FIG. 3D).

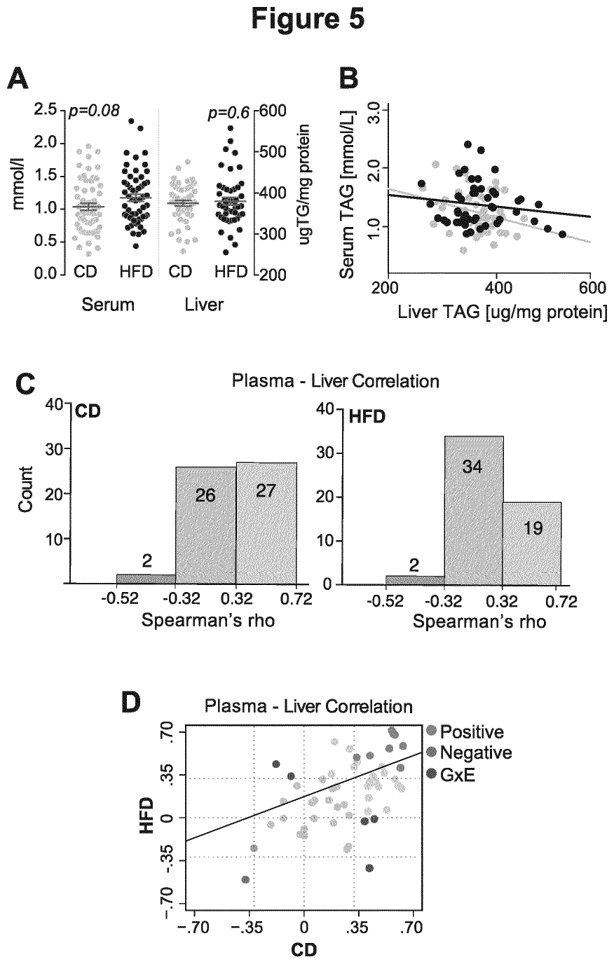

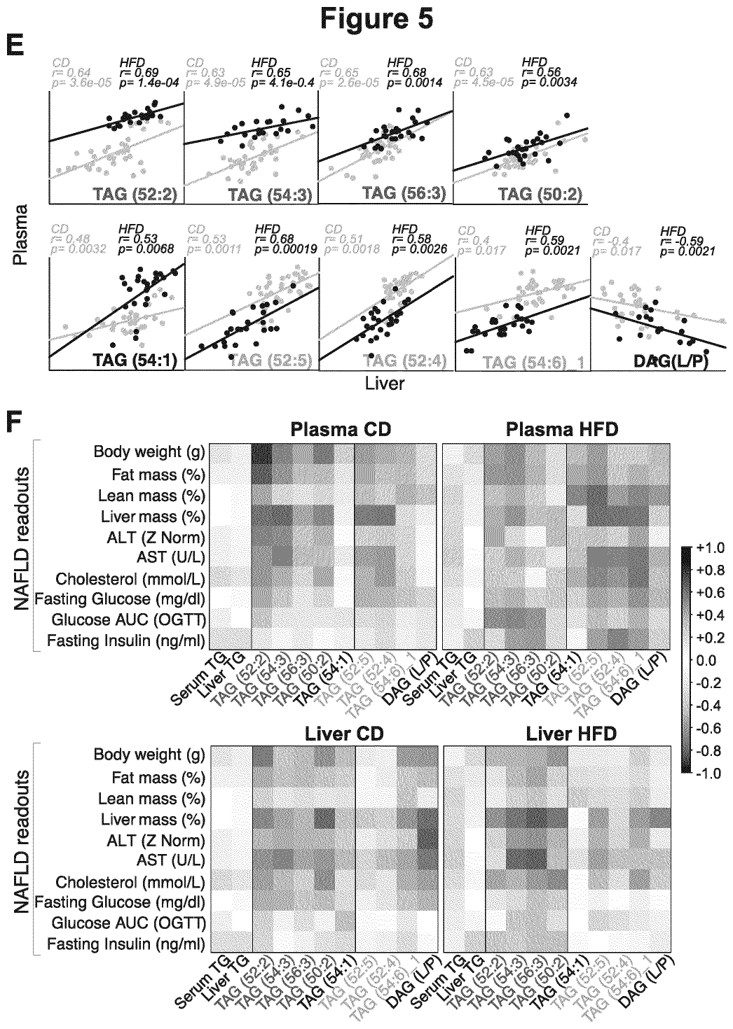

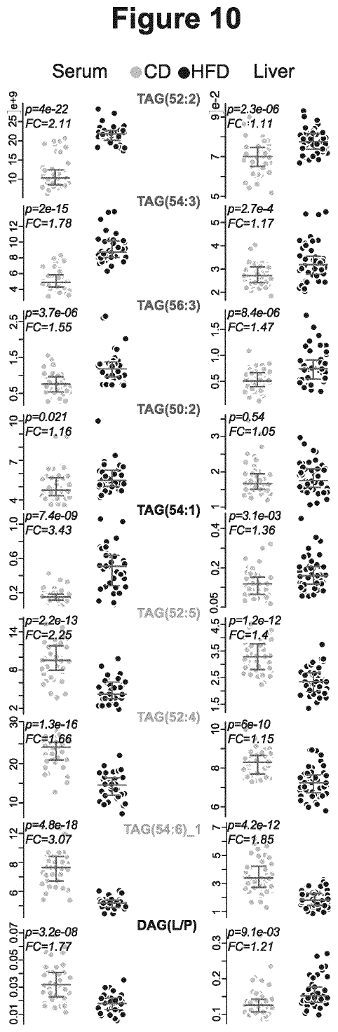

Plasma and liver triglyceride levels are highly variable even across different mice strains and are not always increased under HFD indicating an underlying genetic regulation of triglyceride levels (Champy et al., 2004; Kirk et al., 1995; Lin et al., 2005). In line, plasma or liver TAG levels were highly variable and invariant between CD and HFD in the genetically diverse BXD cohorts (FIG. 5A). Moreover, the liver and plasma TAG levels did not correlate (FIG. 5B). Therefore, it was sought to identify lipid species in plasma, which are representative of their levels in liver. Of the 55 common lipids measured in plasma and liver, 29 in CD and 21 in HFD correlated with a Spearman's rho >±0.32 (FIG. 5C, Table 2E), with 28 lipid species in CD and 13 in HFD being significantly correlated (p<0.05) between liver and plasma (Table 2E). Additionally, there was a strong positive correlation between plasma and liver lipids across both diets (FIG. 5D). In particular, plasma and liver values of 9 of these lipids showed very significant and tight correlations across both diets (FIG. 5D; dark green and orange dots), implying that the levels of these lipids in plasma are a strong reflection of their levels in liver. Pearson correlation of these 9 lipid species (identified in panel 5D) in liver and plasma was again significant in both diets (FIG. 5E, S5). Four of these lipid species (TAG(52:2), TAG(54:3), TAG(56:3) and TAG(54:1)) were significantly increased in the HFD cohorts in both plasma and liver, while three (TAG(52:5), TAG(52:4) and TAG(54:6) were significantly increased in the CD cohorts in both plasma and liver (FIG. 5E and S5). TAG(50:2) was significantly increased in HFD in plasma but not in liver (p-val.=0.54) (FIG. 5E and S5). While DAG(L/P) (DAG(18:2n6/16:0)) showed negative correlation between liver and plasma, because under HFD, it was decreased in plasma, whereas it was increased in liver (FIG. 10). The accumulation of DAG(L/P) in liver and reduction in serum, suggests that its release from liver into the plasma may have been greatly minimised. These 9 lipids, which correlate very strongly in their levels between plasma and liver, could hence be good plasma biomarkers of liver lipid content and serve as a signature of steatosis/NAFLD in plasma.

Correlation of these lipid species, alongside total TAG concentration in liver and plasma, was performed with a number of phenotypes linked with NAFLD including, fasting Insulin, glucose, cholesterol, alanine transaminase (ALT), aspartate transaminase (AST), fat mass, liver mass and body weight (FIG. 5F). For either diet, the total TAG concentration in plasma or liver did not correlate with any of the NAFLD associated phenotypes (FIG. 5F). The TAGs: 52:2, 54:3, 56:3 and 50:2, showed a positive correlation with the NAFLD readouts in both diets in liver and plasma (FIG. 5F, red font). Of note, these lipids were enriched in oleic acid as a side chain (Table 2A). Other TAGs: 52:5, 52:4 and 54:6_1 showed negative correlation with the NAFLD readouts in both diets in liver and plasma (FIG. 5F, blue font). These lipids were enriched in linoleic acid as a side chain (Table 2A). Importantly, these 7 lipids (FIG. 5F, red and blue font) have high h² (55-84%) in plasma in both CD and HFD cohorts (Table 2D). Of note, DAG(L/P), which shows a negative correlation (as well as enrichment) between serum and liver, is the only lipid among the 9 lipids showing an opposite correlation with NAFLD phenotypes in liver and plasma (FIG. 5F, S5). In addition, its h² in plasma was also relatively low compared to the above 7 markers (46% in CD, 32% in HFD; Table 2D). TAG(54:1) can be considered as a diet specific NAFLD marker since it correlated positively with NAFLD markers in CD and negatively in HFD, indicative of a GxE effect, where HFD effect reverses the genetic effect. Its h² in CD being 57% and in HFD, 48% (Table 2D). The unexplained variance for both TAG(54:1) and DAG(L/P) was 35% and 48% respectively (Table 2D), suggesting that these two lipids may not prove to be reliable plasma biomarkers for NAFLD. These findings suggest that TAG(52:2), TAG(54:3), TAG(56:3) and TAG(50:2) are pro-NAFLD markers, whereas TAG(52:5), TAG(52:4) and TAG(54:6)_1 are anti-NAFLD markers irrespective of the diet. A previous study (Chitraju et al., 2012) suggested 4 TAG species (52:2, 54:1, 52:1, and 56:2) as biomarkers of hepatic steatosis in mice, based on their increased levels in hepatic lipid droplets in C57BL6/J mice fed a HFD. Lipidome phenotyping of the present disclosure not only confirmed the increased levels of these lipids in livers, but also in plasma (Table 2A, data not shown for liver). However, of these 4 lipids, only TAG(52:2) was amongst 9 lipids disclosed herein showing strong correlation across diets in both plasma and liver and with NAFLD readouts (FIG. 5D, green dots; FIG. 5F). Moreover, the relevance of the other three lipids (54:1, 52:1 and 56:2) as NAFLD biomarkers could only be established due to poor/no correlation with NAFLD readouts (FIG. 7). This finding signifies the importance of having a strong coherence between plasma and liver lipids in order for it to be established as a biomarker, as demonstrated by the 7 biomarkers here (FIG. 5F; red and blue font). Additionally, the data herein disclosed illustrate the importance of understanding the physiological relevance of individual lipid species rather than the whole class.

Validation of Lipid Species as Biomarkers of NAFLD and Metabolic Fitness in Mice and Humans.

It was next validated the lipid biomarkers of NAFLD and metabolic fitness identified in FIG. 5J and FIG. 2C-D in a mouse model of high-fat high-sucrose (HFHS) diet induced fatty liver (Gariani et al., 2016). C57BL6/J mice were fed with HFHS for 18 weeks. Another cohort was given nicotinamide riboside (NR; shown to ameliorate fatty liver [ref]) supplemented HFHS diet for 18 weeks (preventive treatment) and the other was given NR supplemented HFHS diet after 9 weeks on HFHS diet (therapeutic treatment). To further explore the relevance of these findings across the whole NAFLD spectrum (fatty liver, fibrosis/NASH and cirrhosis) in human subjects, it was analyzed the plasma lipidome of patients from all the 3 stages of NAFLD.

In this disclosure, it was used a systems genetics approach—consisting of a multilayered omics (genetics, lipidomics and phenomics) analysis—to examine the plasma lipidome in the mouse BXD GRP. For the 129 distinct lipid species measured, it was demonstrated the dietary footprint on lipid species, their interaction and their impact on metabolic traits. It was then examined how genetic, environmental and GxE factors influence lipid species and identified genetic loci that may drive changes for the 112 of 129 lipid species which mapped to QTLs. Subsequently 65 of these lipid species QTLs were linked to GWAS genes associated with blood lipids. Finally, 7 lipid species were identified as highly predictive plasma biomarkers reflective of NAFLD and their relevance was validated in a mouse model of fatty liver disease and in human subjects with various severity of NAFLD. While it was earlier (Chick et al., 2016; Hui et al., 2015; Williams et al., 2016; Wu et al., 2014) used the mouse GRP to study different omics layers, including transcriptomics, proteomics, and metabolomics, this is the first disclosure to quantify individual lipid species across classes in any mouse population (129 lipids in 271 mice from 78 cohorts) and provide the physiological impact of these lipids. Additionally, though human studies have profiled plasma (Gorden et al., 2015; Oresic et al., 2013; Puri et al., 2009) and liver lipids (Puri et al., 2007) separately from NAFLD patients, it is important to note that an integrated study elucidating the representative lipids of liver in plasma as a biomarker has not been performed.

Using different bioinformatics approaches, it was shown that majority of the lipids species are highly correlated and co-regulated, both within and across different 10 lipid classes measured. This resonates the underlying fact that any change in a lipid metabolic pathway induced by an experimental or physiological intervention will almost assuredly result in compensatory changes in other pathways affecting discrete lipid pools (Farese et al., 2012). In order to reduce the complexity of the lipids measured, it was used an unbiased network method—WGCNA—to group lipids in modules/clusters. Though the modules were primarily enriched in lipids from the same class, they also contained lipids from other classes, reiterating the co-regulation and interconnectivity of lipid species from different classes. The analysis herein disclosed shows that within the same lipid class, some lipid species are associated with healthy metabolic traits and other with unhealthy metabolic traits, providing testimony to the fact that all lipid species in a class do not have the same physiological impact (Farese et al., 2012). Therefore, entire classes of lipids should not be categorized as good or bad lipids, yet until recently more granular measurements of specific lipid species have been prohibitively difficult (Han, 2016; Hyotylainen and Oresic, 2014, 2015). In both research and clinical diagnosis, lipids have been predominantly measured only as the sum of total lipid classes (e.g. total TAG, total cholesterol, total phospholipids), which results in measurements that are skewed by the most abundant lipids in each particular class. These conglomerate lipid measurements can be misleading in terms of their physiological impact, as the data herein disclosed show that many of the less abundant species are good predictors of metabolic health than the most abundant ones.

The change in diet from chow diet (6% calories from fat) to high fat diet (60% calories from fat) had a significant impact on nearly all plasma lipid species measured—73%. Likewise, even within a dietary set, all lipids had significantly different regulation between the strains with the lowest and highest levels. Across all conditions, roughly 50% of variation could be attributed to diet-independent genetic factors, with an additional ˜10% of variation being attributable to uniform diet-induced changes, or a strain-dependent response to dietary differences. Together, the present disclosure can explain more than half of the observed variation in plasma lipid levels for 85% of the 129 measured lipid species. For the variation attributable to genetic or GxE factors, it was identified over 200 novel lipid QTLs containing known and novel putative regulators of lipid metabolism. Taking advantage of the known human GWAS loci/genes associated with blood lipid levels and associated traits; it was gone one step ahead and uncovered via a cross-species extrapolation, the association between these human genes and the lipid species identified in BXD study of the present disclosure.

This demonstrated the association between 65 lipid species in the BXDs and 32 human loci for blood lipids, including 7 syntenic regions in both mice and humans. This link underscores the power of mouse GRP studies to not only complement human genetic studies in a more refined manner, but also to find novel loci/genes controlling lipid species levels and to provide a proper setting for mechanistic studies.

Triglycerides are often considered as a signature of the metabolic syndrome, including NAFLD, despite a very wide interindividual difference in its levels (Cohen et al., 2011; Johansen et al., 2011; Listenberger et al., 2003). In line, it was not seen any significant difference in plasma or liver triglyceride levels between the diets, suggesting a strong underlying genetic control in modulating triglyceride levels. This goes in line with numerous human GWAS, which have identified at least 32 loci associated with only triglyceride levels (Teslovich et al., 2010). Furthermore, liver and plasma triglycerides did not correlate indicating that plasma triglyceride levels do not reflect liver triglyceride levels always (Romeo et al., 2008). By comparing the lipidome of serum (this disclosure) and liver (Jha et al. accompanying paper), 55 common lipids were identified, of which only 9 in plasma (8 TAGs and 1 DAGs) were a reflection of their levels in liver. Importantly, 4 of these lipid species were identified as pro-NAFLD biomarkers and 3 as anti-NAFLD biomarkers. Some of these biomarkers could separately be verified in both mice and human NAFLD subjects. The identification of these biomarkers using mouse GRP and validation in humans underscores the potential of mouse population genetics for translational research. While currently the diagnosis of NAFLD relies on invasive liver biopsies, which are also discretionary in nature and relying on histological scoring (Chalasani et al., 2012); MS-based lipidomics analysis coupled with plasma biochemistry markers may prove to be a more forward looking approach, which is non-invasive, less labor intensive and standardized. These plasma biomarkers also offer a chance to monitor disease progression and efficacies of preventive or therapeutic interventions.

This disclosure uncovers the potential of lipidomics combined with systems genetics to identify powerful biomarkers for heath and disease. The wealth of information on novel lipid QTLs and the phenotypic footprint of these lipid species provide a robust resource to the scientific community for in-silico data analysis. Further, the identification of lipid species as plasma biomarkers reflective of NAFLD from mice to humans strengthen the fact that lipid species can be used as powerful biomarkers of metabolic health and disease. Improved lipid analysis will benefit both molecular medicine and nutritional research, as human health is clearly dependent on human diet and genetics.

Validation of Lipid Species as Biomarkers of NAFLD and Metabolic Fitness in Mice and Humans.

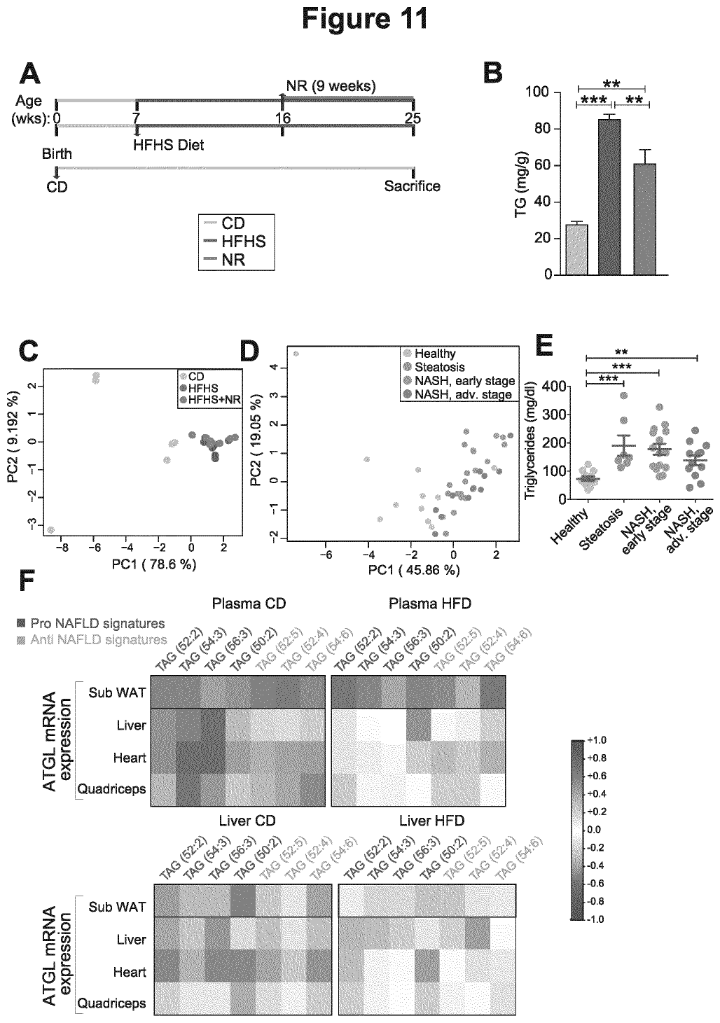

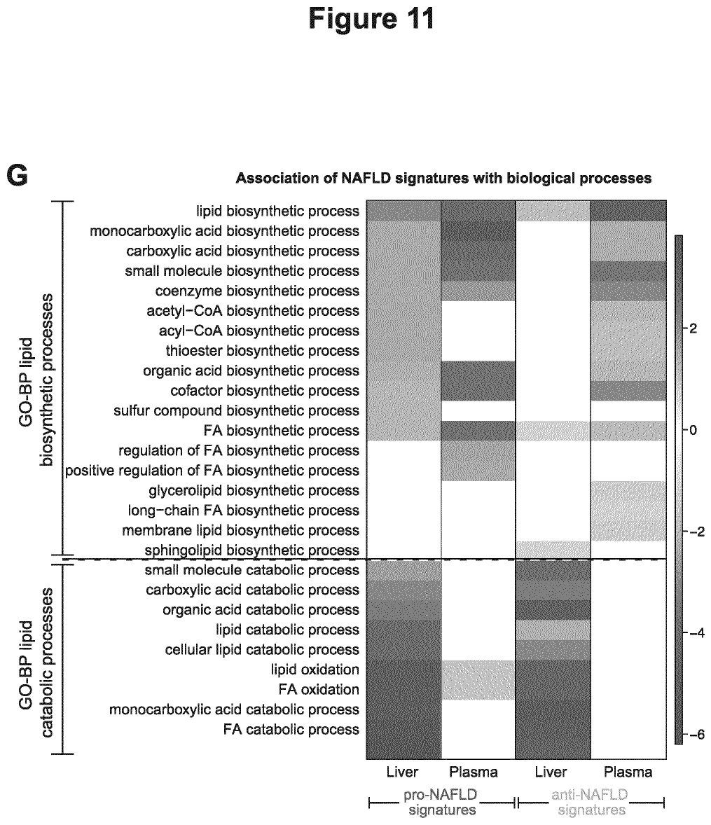

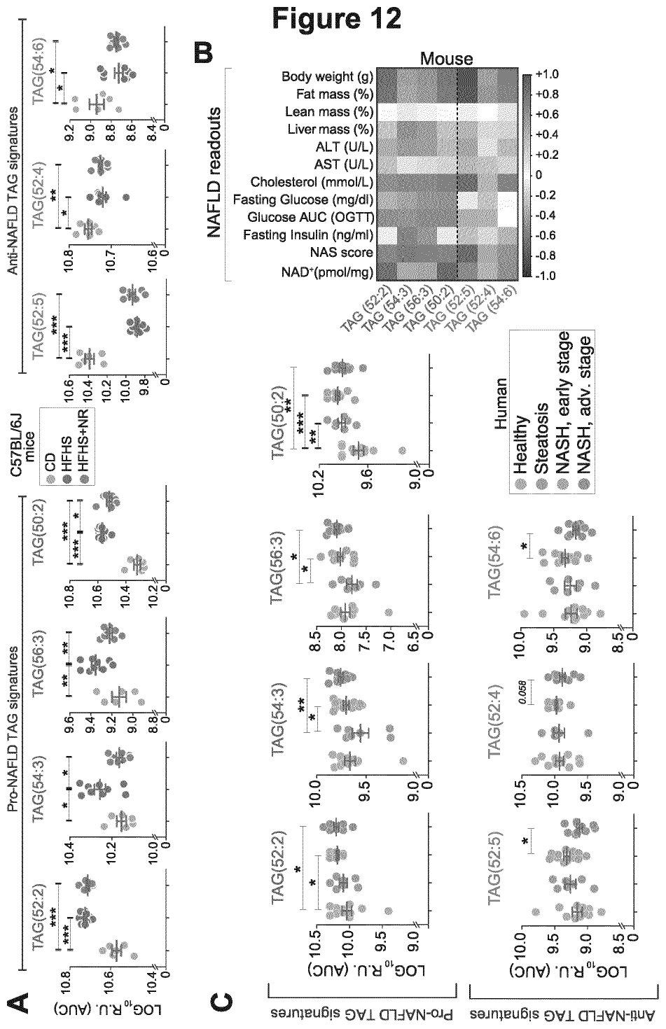

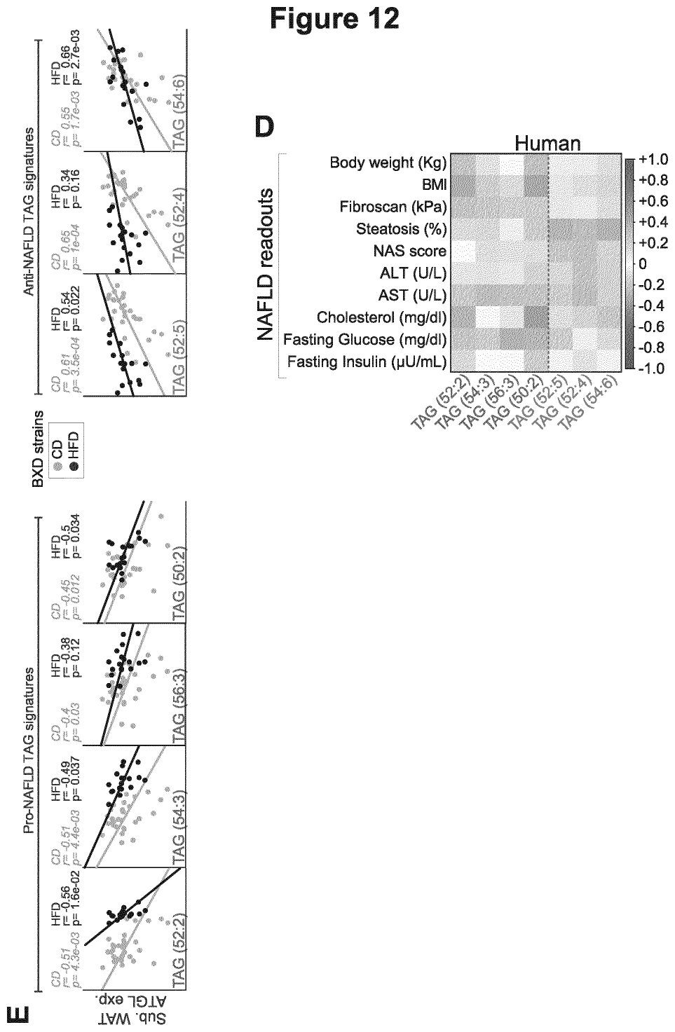

It was then tested the relevance of these lipids as signatures of fatty liver in a different mouse model of NAFLD (induced by high-fat high-sucrose (HFHS) diet) and tested whether a NAFLD lowering therapeutic intervention, i.e. nicotinamide adenine dinucleotide (NAD⁺) precursor, nicotinamide riboside (NR), impacts these lipid markers. As such, the liver lipids were compared of mice fed CD or HFHS diet for 18 weeks, or mice that were fed HFHS diet supplemented with NR 9 weeks after the start of HFHS diet when they had already developed NAFLD (a therapeutic intervention, HFHS+NR) (FIG. 11A). Though liver total TAG concentration was significantly elevated in the HFHS group and decreased by NR (FIG. 11B), all individual TAG species did not show this profile despite their uniform genetic background (C57BL/6J) (FIG. 11C). Of all the TAG species measured, only 53% were increased in HFHS cohort while 33% were decreased and 14% unchanged (data not shown). Importantly, all four pro-NAFLD lipids (TAGs 52:2, 54:3, 56:3 and 50:2) from the BXD HFD study (FIG. 5F) were also increased in C57BL6/J mice fed HFHS diet, whereas the three anti-NAFLD lipids (TAGs 52:5, 52:4 and 54:6) were decreased in HFHS diet cohorts (FIG. 12A). NR significantly lowered three of the four pro-NAFLD lipids (TAGs 54:3, 56:3, 50:2), but increased the anti-NAFLD lipids only to a slight extent (FIG. 12A). Importantly, the pro-NAFLD lipids correlated positively, whereas the anti-NAFLD lipids correlated negatively with clinical NAFLD readouts (FIG. 12B). Notably, the liver NAD⁺ levels correlated negatively with the pro-NAFLD lipids, and positively with anti-NAFLD lipids (FIG. 12B), showing that NAD⁺ levels are depleted in steatotic livers and are replenished after NR treatment.

To further explore the clinical relevance of these findings, the plasma lipid species of healthy patients, patients with steatosis, early stage NASH (mild to moderate fibrosis (F1+F2)) and advanced stage NASH (severe bridging fibrosis or cirrhosis (F3+F4)) were analyzed. The first dimension (PC1) of the PCA of 55 TAG species segregated nearly all of the healthy individuals from the steatosis and NASH group (FIG. 11D). However, the variance explained by the PC1 was only 46%, implying that the TAG profile in humans is highly variable possibly due to the high genetic variation (FIG. 11D). Despite the significant differences in total TAG plasma levels between the healthy and NAFLD groups (FIG. 11E), only a few individual TAG species were significantly changed, which is in line with the findings from mice. Only 20% of TAG species were increased in steatosis vs. healthy, while 64% increased in early stage NASH vs. healthy and 49% increased in advanced stage NASH vs. healthy group (data not shown). However, the pro-NAFLD lipids were increased in steatosis and/or the two NASH groups. Compared to the healthy group, TAG(50:2) was increased in both steatosis and the two NASH groups (F1+F2 and F3+F4), while TAG(52:2) was increased only in the two NASH groups; whereas TAG(54:3) and TAG(56:3) were increased in the two NASH vs. the steatosis groups (FIG. 12C). Conversely, the three anti-NAFLD lipids, TAG(52:5), TAG(52:4) and TAG(54:6), were decreased in advanced stage compared to early stage NASH (FIG. 12C). In line, the pro-NAFLD lipids correlated positively with NAFLD readouts whereas, the anti-NAFLD markers correlated negatively (FIG. 12D). Taken together, these data confirm the findings from the BXDs in a different model of diet-induced NAFLD in mice as well as in humans, indicating that these lipid species may be a more universal signature of NAFLD across different diets and also relevant in humans.

In order to obtain insight on the affinity of TAG-metabolizing enzymes for the NAFLD signatures, the association of adipose triglyceride lipase (ATGL)—the rate limiting TAG-metabolizing enzyme—with these NAFLD TAG signatures was tested. Atgl expression in white adipose tissue (WAT) negatively correlated with the pro-NAFLD signatures and positively correlated with the anti-NAFLD signatures in both CD and HFD cohorts (FIG. 12E). This correlation was also observed with the expression of Atgl in other metabolic tissues (liver, heart and muscle) (FIG. 11F), however, the strongest association was observed with WAT Atgl expression, since Atgl lipase activity is ˜10 times higher in WAT compared to other tissues. These findings are consistent with inventors previous experimental findings showing that Atgl is important to provide protection from steatohepatitis and that Atgl-KO mice are susceptible to develop NAFLD/NASH. Additionally, the pro-NAFLD signatures in both plasma and liver correlated positively with lipid biosynthetic pathways and negatively with oxidative pathways (FIG. 11G). Conversely the anti-NAFLD signatures correlated positively with oxidative pathways and negatively with lipid biosynthetic pathways (FIG. 11G). Taken together, these biological corroborations of the TAG signatures provide proof of concept validation of the lipid species measured and validate the usefulness of the resource.

Identification of Cardiolipin Species as Signatures of Healthy and Fatty Liver

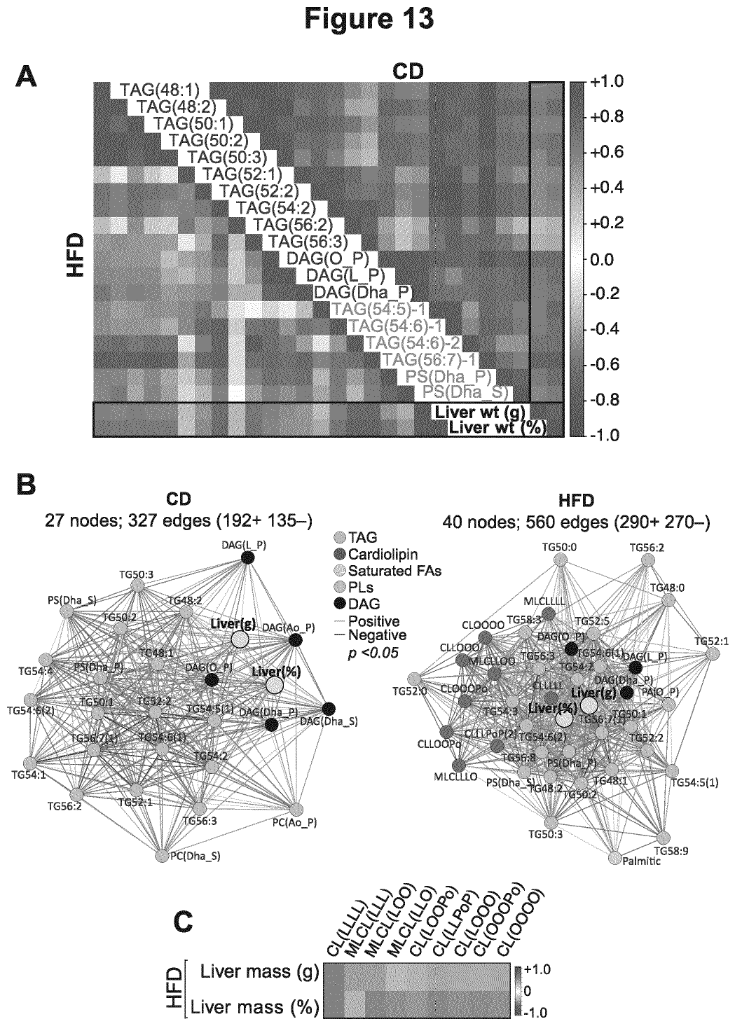

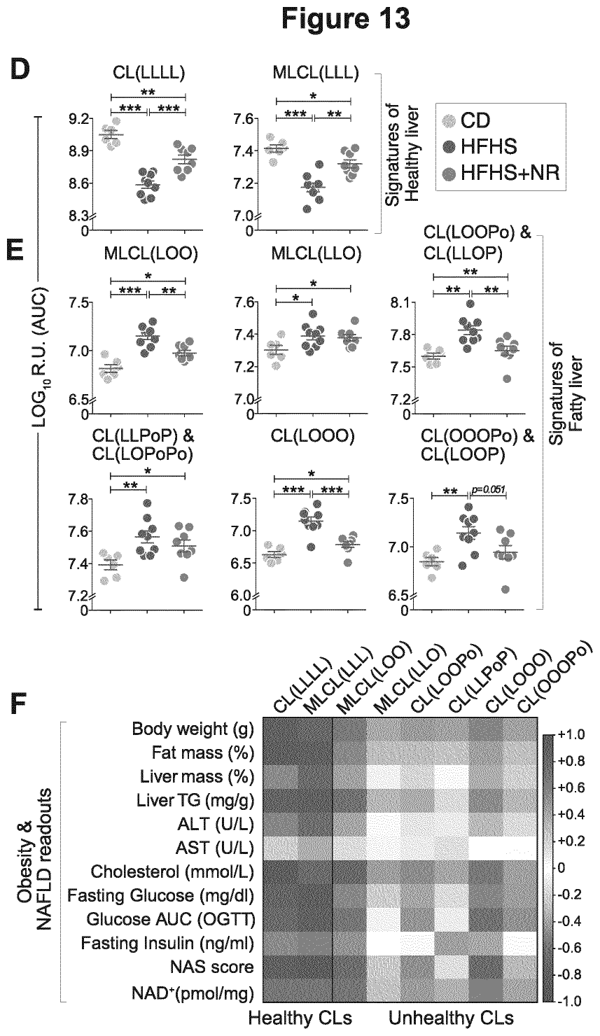

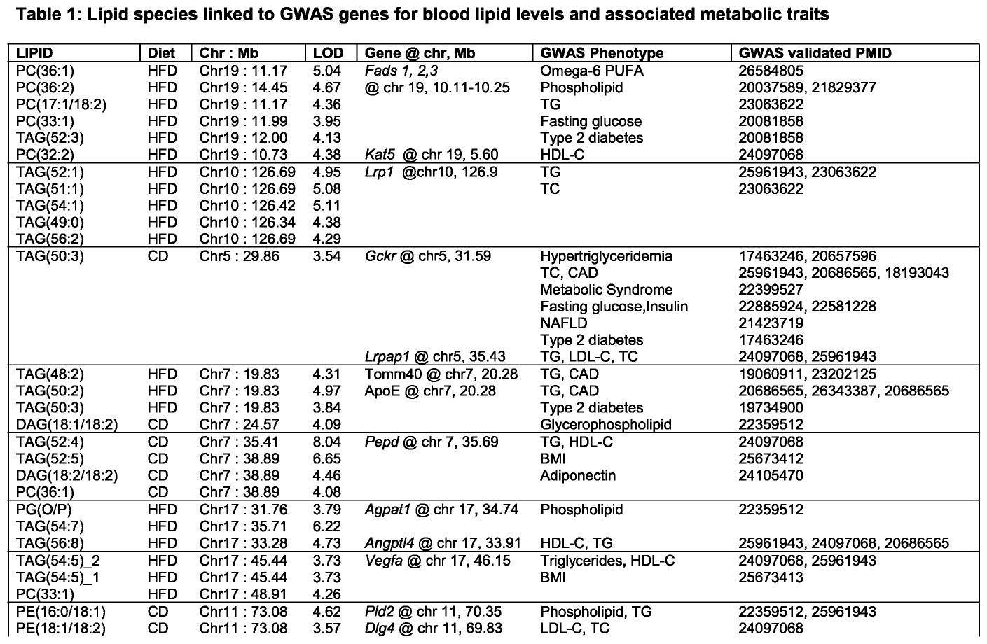

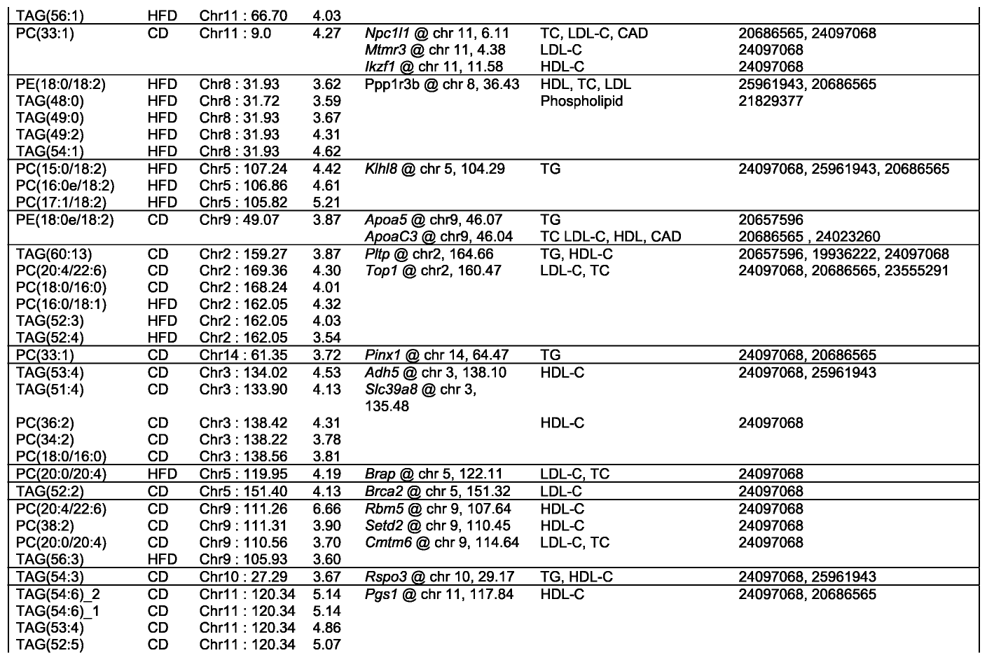

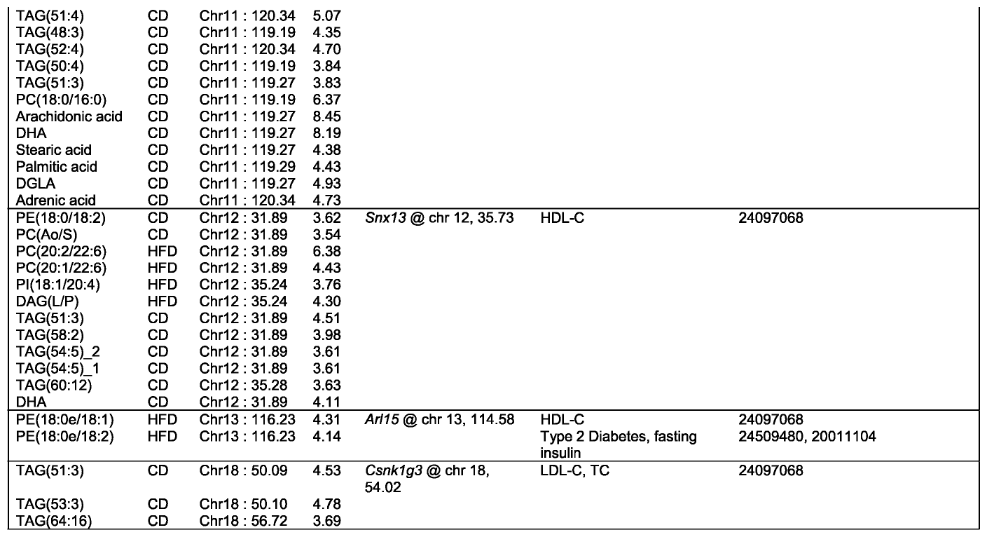

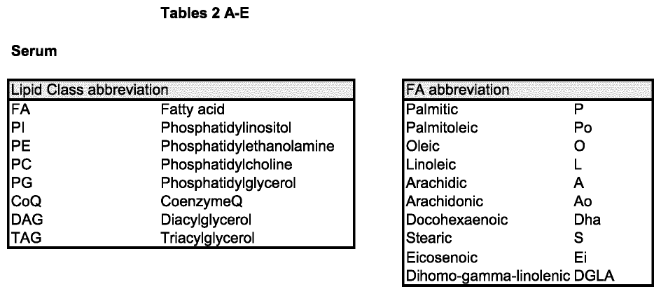

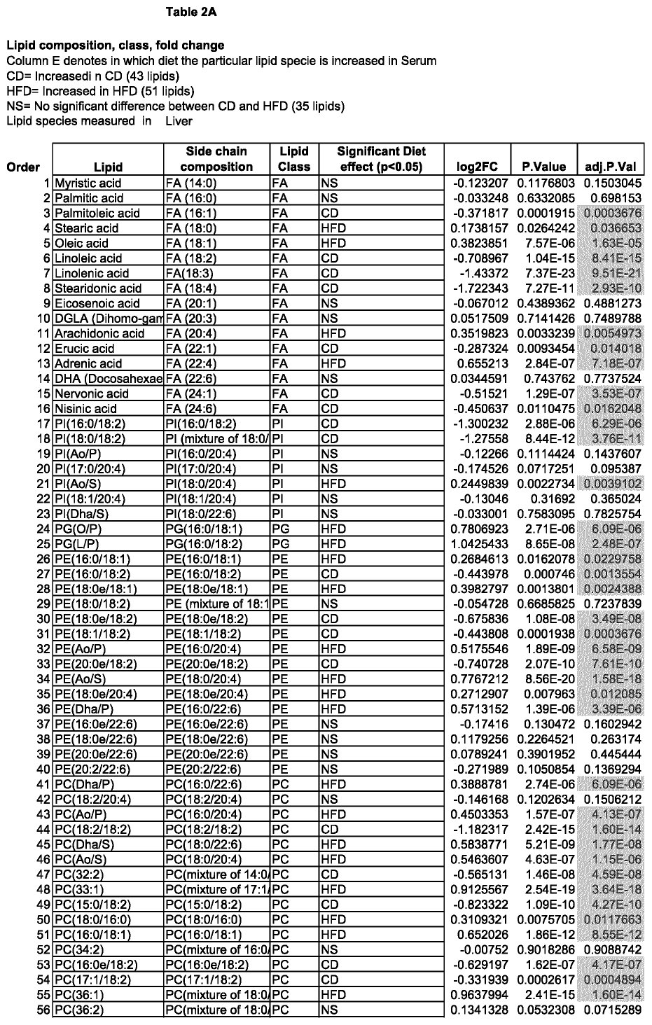

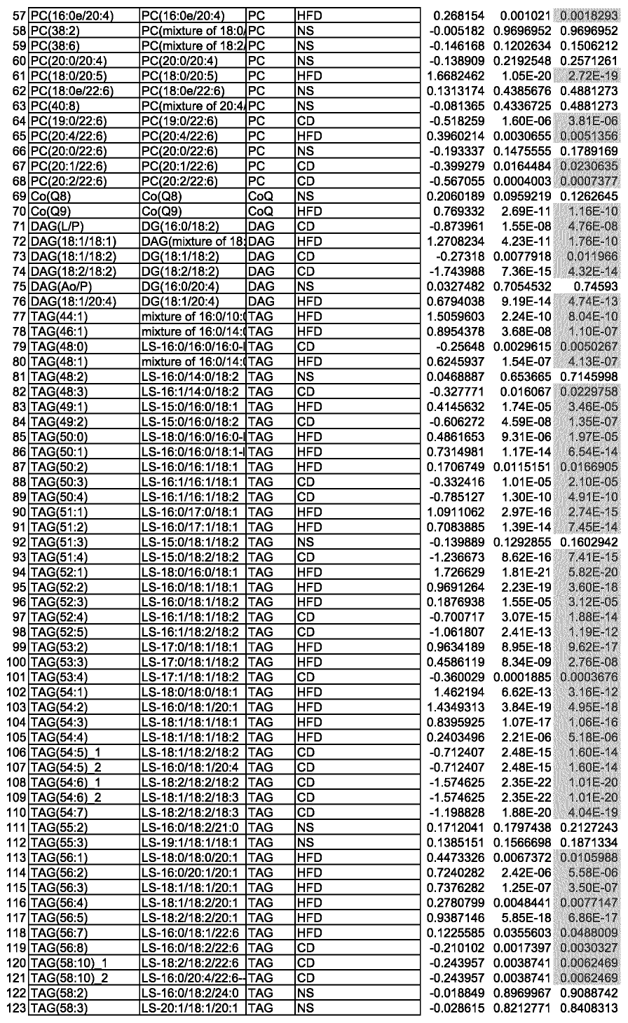

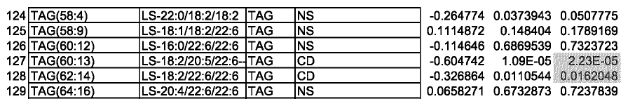

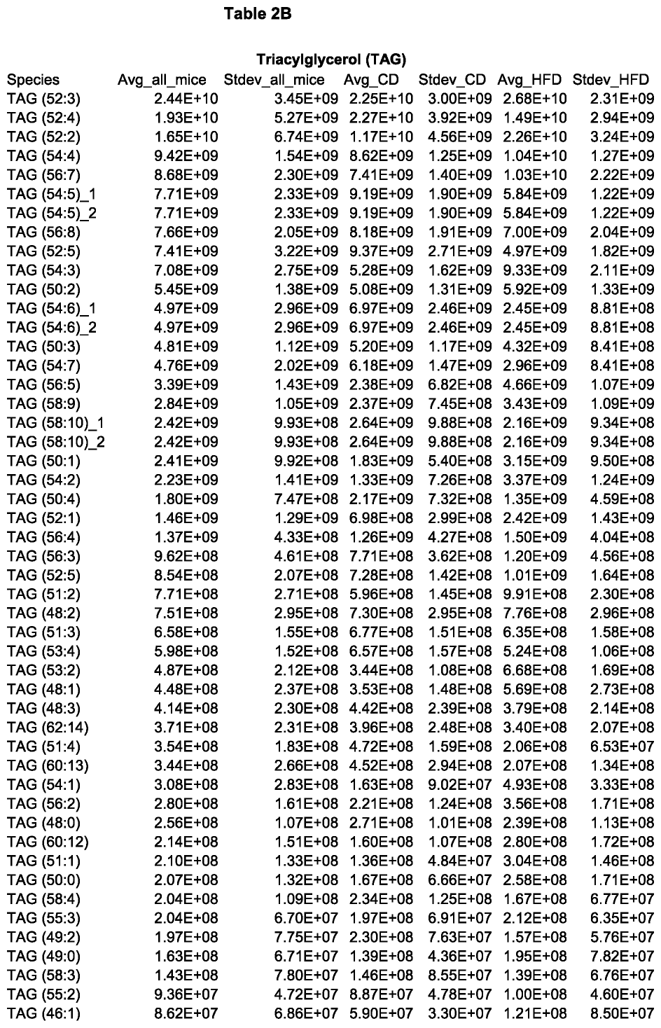

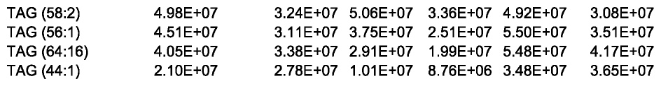

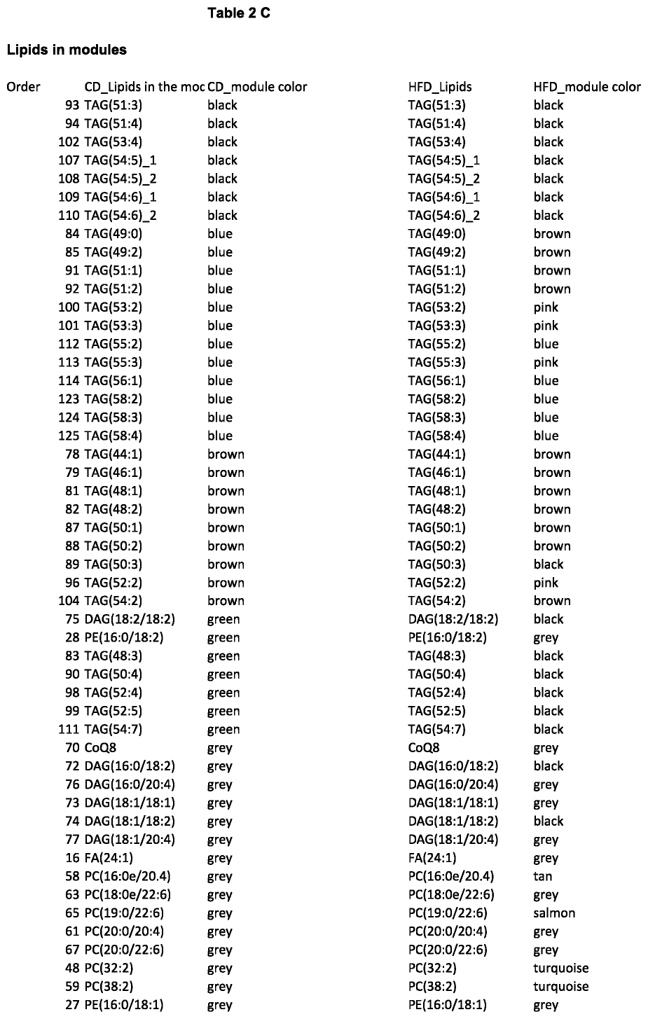

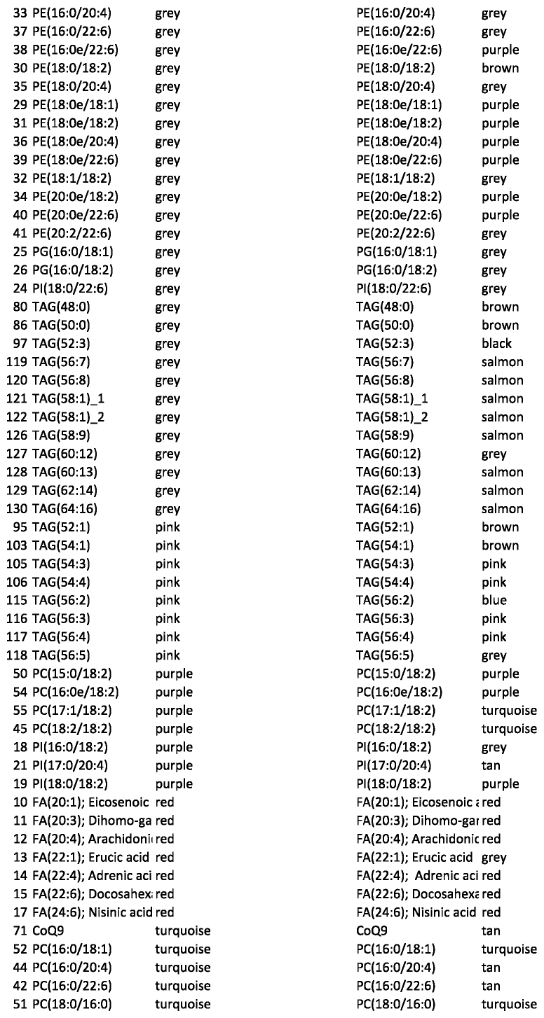

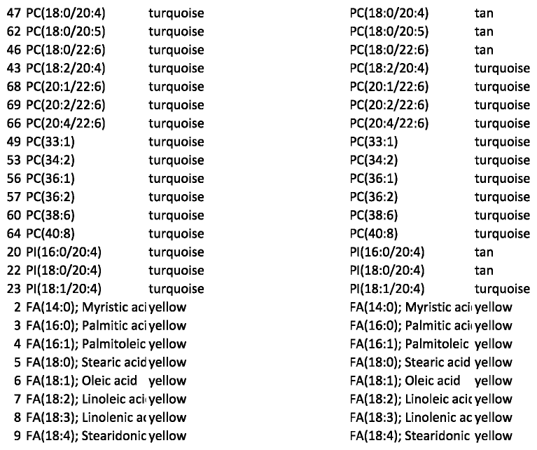

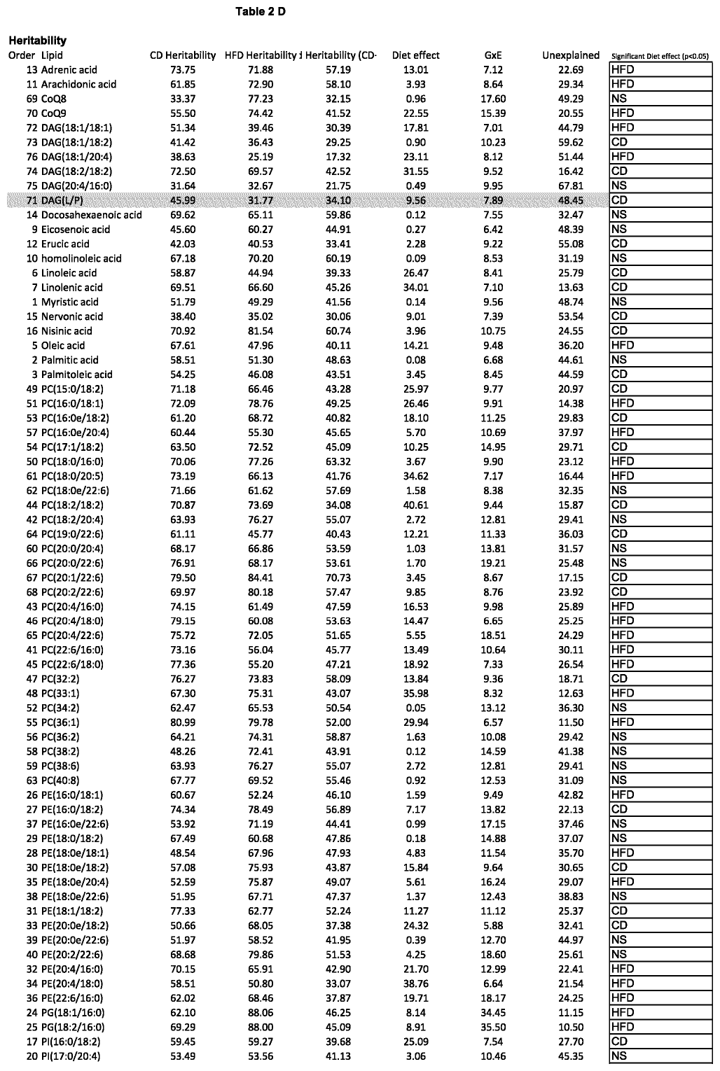

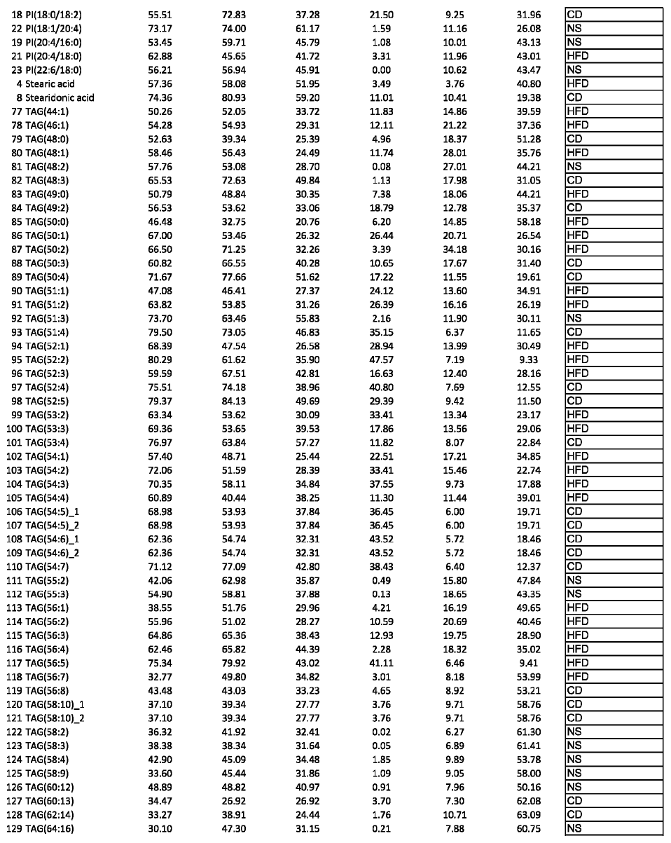

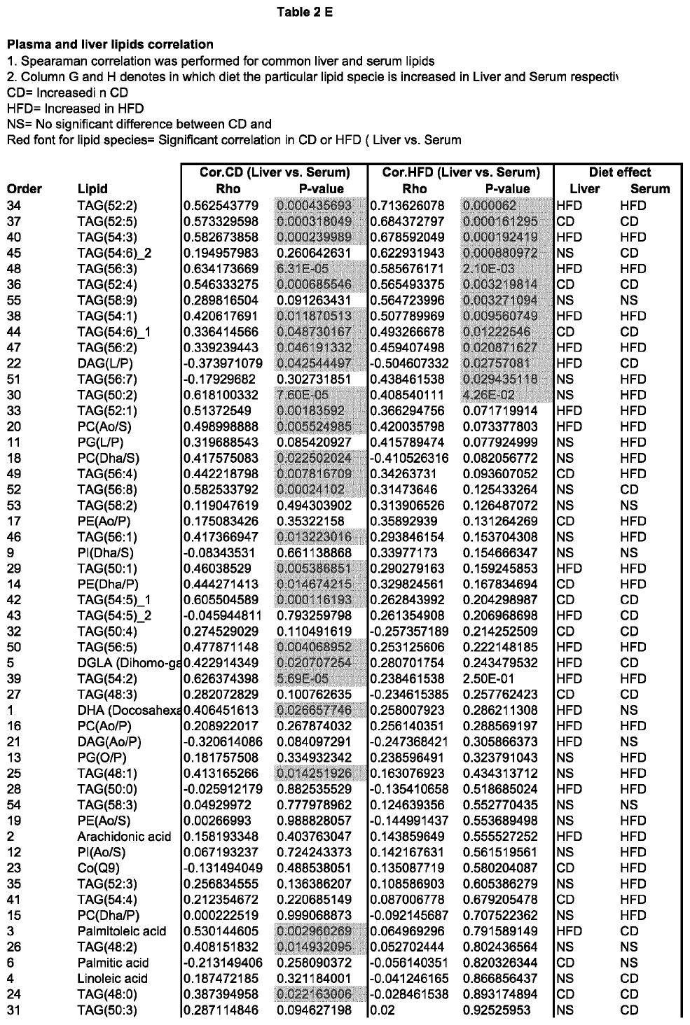

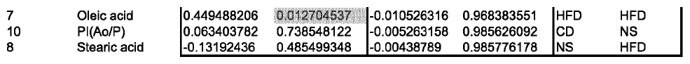

From all lipids measured, two clusters of lipid species with strong diet-independent association with liver mass were identified (FIG. 13A). A cluster of 13 species composed of TAGs and DAGs with a low degree of unsaturation (1-3 double bonds; dominated by lipids from the black and brown modules) correlated positively with liver mass (FIG. 13A; green font); whereas, another cluster of 6 lipids comprising highly unsaturated TAGs (6-7 double bonds; dominated by lipids from the purple module) along with two phosphatidylserine species, correlated negatively with liver mass (FIG. 13A; orange font). Since the diet changed the landscape of most liver lipids, lipids that strongly associated with liver mass in each individual diet were next analysed (FIG. 13B). 27 lipids in CD and 40 in HFD (including all the 19 lipids from FIG. 13A) strongly correlated with liver mass. Of note, liver mass was centrally positioned in the resulting HFD network showing dense correlations with the 40 lipid species compared to the CD network where liver mass was at the periphery of the network with 27 lipids (FIG. 13B). Interestingly, a subset of nine CL and monolyso-CL (MLCL) species showed a predominant association with liver mass in HFD, but not in CD (FIG. 13B; red nodes). This finding is noteworthy because CL—the signature phospholipid of the mitochondrial inner membrane—is indispensable for a range of mitochondrial activities. Alterations in the content and/or structure of CL have been reported in several tissues in a variety of pathological settings. However, a major unresolved question is whether CL molecules with different acyl chain compositions differ functionally. Of these nine species, only tetralinoleoyl-CL (CL(LLLL)) and its precursor/remodeling intermediate, trilinoleoyl-MLCL ((MLCL(LLL)) (neither belonging to any module) showed negative correlation with liver mass; whereas the other seven CLs enriched in monounsaturated FAs (MUFA), oleic (O) and palmitoleic (Po) acid (all from the yellow module), positively correlated with liver mass (FIG. 13C). This demonstrates a change in CL remodeling under HFD that depletes the CL predominant in healthy tissue—CL(LLLL), and its precursor, MLCL(LLL)—suggesting that these CL species may be signatures of healthy/normal liver. Conversely, the other seven MUFA enriched CLs that correlated positively with liver mass in HFD may be considered signatures of unhealthy/fatty liver.

It was next tested whether this change in the profile of nine CL species is a general phenomenon in other dietary-induced models of hepatic steatosis and mitochondrial dysfunction and if the profile can be reverted by ameliorating hepato-steatosis via enhancing mitochondrial function. It has been previously shown that nicotinamide riboside (NR) treatment ameliorates high-fat high-sucrose (HFHS) diet induced fatty liver disease by boosting nicotinamide adenine dinucleotide (NAD+) levels and thereby enhancing mitochondrial function. Therefore, lipidomic profiling from the livers of C57BL/6J mice—the most commonly used laboratory mouse strain—fed on (i) CD or (ii) HFHS diet for 18 weeks or (iii) HFHS+nicotinamide riboside (NR), added 9 weeks after the start of the HFHS diet (therapeutic approach) was performed. HFHS diet decreased the CL signatures of healthy/normal liver—CL(LLLL) and MLCL(LLL)—whereas it increased the six CL signatures of unhealthy/fatty liver—MLCL(LOO), MLCL(LLO), CL(LOOPo), CL(LLPoP), CL(LOOO), CL(OOOP)—enriched in MUFAs (FIGS. 13D and 13E). [The CL, CL(OOOO), detected in the BXD study (FIG. 136/13C), was too low to be detected in all samples from the NR study]. Interestingly, NR treatment increased the levels of the healthy CLs, whereas it decreased the levels of the unhealthy CLs (FIGS. 13D and 13E). Importantly, the two healthy CLs correlated negatively with obesity and NAFLD traits while the unhealthy CLs showed positive correlation (FIG. 13F). These data show that all lipid species within a class do not behave similarly, as demonstrated here with specific CL species that have signatures of healthy or fatty liver.

The invention relates to liver lipid species selected from the triacylglycerol (TAG) and cardiolipin (CL) lipid classes measured by LC-MS/MS and use thereof as diagnostic and prognostic biomarkers of fatty liver. The CL species, enriched in polyunsaturated linoleic (L) acid, including its precursor/remodeling intermediate monolysocardiolipins (MLCL): CL(LLLL) and MLCL(LLL) and the TAG species: TAG(52:5), TAG(52:4) and TAG(54:6) are biomarkers of a healthy liver and are decreased in the presence of fat accumulation. Whereas, the CL species: enriched in monounsaturated fatty acids—oleic (O) and palmitoleic (Po) acid—including MLCL(LOO), MLCL(LLO), CL(LOOPo), CL(LLPoP), CL(LOOO), CL(OOOP) and the TAG species: TAG(52:2), TAG(54:3), TAG(56:3) and TAG(50:2) are biomarkers of unhealthy liver and are increased in the presence of fat accumulation. These lipid species are hence reliable biomarkers to diagnose various diseases with fatty liver, including non-alcoholic fatty liver disease (NAFLD), alcoholic fatty liver disease (AFLD), non-alcoholic steatohepatitis (NASH), alcoholic steatohepatitis (ASH); but also other diseases that are typified by liver fat accumulation. Moreover, the diagnostic precision can be increased by the combined profile of these 5 positive (beneficial) and 10 negative (harmful) biomarkers. These quantitative biomarkers will facilitate the diagnosis of these diseases, which now relies on liver biopsies coupled with discretionary histological analysis, resulting in their “under-” or “mis-diagnosis” and often a late start of treatment. Furthermore, they can be used to monitor the efficacy of preventive and therapeutic measures to lower liver fat content.

CL and MLCL species are usually detectable only in liver and therefore their biomarker relevance is limited to liver, whereas the TAG species are present in both liver and plasma and therefore their biomarker utility is applicable to both plasma and liver. Thus specifically (i) in plasma, the TAG species: TAG(52:2), TAG(54:3), TAG(56:3) and TAG(50:2) are biomarkers of an unhealthy liver and are increased in the plasma in the presence of a fatty liver, while the TAG species: TAG(52:5), TAG(52:4) and TAG(54:6) are biomarkers of a healthy liver and are decreased in the presence of a fatty liver; (ii) in liver, the CL species, enriched in polyunsaturated linoleic (L) acid, including its precursor/remodeling intermediate monolysocardiolipins (MLCL): CL(LLLL) and MLCL(LLL) and the TAG species: TAG(52:5), TAG(52:4) and TAG(54:6) are biomarkers of a healthy liver and are decreased in the presence of fat accumulation, whereas the CL species: enriched in monounsaturated fatty acids—oleic (O) and palmitoleic (Po) acid—including MLCL(LOO), MLCL(LLO), CL(LOOPo), CL(LLPoP), CL(LOOO), CL(OOOP) and the TAG species: TAG(52:2), TAG(54:3), TAG(56:3) and TAG(50:2) are biomarkers of unhealthy liver and are increased in the presence of fat accumulation.

According to an aspect, the present invention provides a method for diagnosing a fatty liver disease or a predisposition therefor in a subject, said method comprising
providing a sample derived from a subject suspected to suffer from a fatty liver disease,
determining in the sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
comparing the amount of the at least one plasma or liver triglyceride (TAG) biomarker, liver cardiolipin (CL) biomarker and/or monolysocardiolipin (MLCL) biomarker with a reference, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for the fatty liver disease.

In a preferred embodiment, the sample is selected from the group comprising liver biopsy (sample), plasma sample or blood cells sample. In most preferred embodiment, the sample is selected from the group comprising liver biopsy (sample) and plasma sample.

Plasma samples are easy to obtain, easy to manipulate, non-invasive, less labor intensive and standardized. They also avoid risk for adverse effects, associated with liver biopsies. In plasma samples only the plasma TAG species of the invention can be detected, whereas the liver CL and MLCL biomarkers cannot be detected; these plasma biomarkers also offer a possibility to monitor disease progression and the efficacy of preventive or therapeutic interventions. In case more specific and/or reliable and/or accurate and/or sensitive results are needed, liver biopsy samples may be needed, where the liver TAG, CL and MLCL biomarkers of the invention can be detected to offer a possibility to monitor disease progression and the efficacy of preventive or therapeutic interventions in liver.

Accoridng to another aspect, the present invention provides a method for diagnosing a fatty liver disease or a predisposition therefor in a subject, said method comprising
providing a plasma or liver sample derived from a subject suspected to suffer from a fatty liver disease,
determining in the plasma or liver sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma or liver triglyceride (TAG) biomarker with a reference, wherein increase of TAG(52:2), TAG(54:3), TAG(56:3), TAG(50:2) or a combination thereof and/or decrease of TAG(52:5), TAG(52:4), TAG(54:6) or a combination thereof is indicative for the fatty liver disease.

According to another aspect, the present invention provides a method for diagnosing a fatty liver disease or a predisposition therefor in a subject, said method comprising
providing a plasma sample derived from a subject suspected to suffer from a fatty liver disease,
determining in the plasma sample of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma triglyceride (TAG) biomarker with a reference, wherein increase of TAG(52:2), TAG(54:3), TAG(56:3), TAG(50:2) or a combination thereof and/or decrease of TAG(52:5), TAG(52:4), TAG(54:6) or a combination thereof is indicative for the fatty liver disease.

According to a further aspect, the present invention provides a method for monitoring the progression of a fatty liver disease in a subject diagnosed to suffer from a fatty liver disease, said method comprising
providing a sample derived from a subject diagnosed to suffer from a fatty liver disease,
determining in the sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
comparing the amount of the at least one plasma or liver triglyceride (TAG) biomarker, liver cardiolipin (CL) biomarker and/or monolysocardiolipin (MLCL) biomarker with a reference, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for the fatty liver disease progression.

In a preferred embodiment, the sample is selected from the group comprising liver biopsy (sample), plasma sample or blood cells sample. In most preferred embodiment, the sample is selected from the group comprising liver biopsy (sample) and plasma sample.

Plasma samples are easy to obtain, easy to manipulate, non-invasive, less labor intensive and standardized. They also avoid risk for adverse effects, associated with liver biopsies. In plasma samples only the plasma TAG species of the invention can be detected; these plasma biomarkers also offer a possibility to monitor disease progression and the efficacy of preventive or therapeutic interventions. In case more specific and/or reliable and/or accurate and/or sensitive results are needed, liver biopsy samples may be needed, where the liver TAG, CL and MLCL biomarkers of the invention can be detected to offer a possibility to monitor disease progression and the efficacy of preventive or therapeutic interventions in liver.

According to a further aspect, the present invention provides a method for monitoring the progression of a fatty liver disease in a subject diagnosed to suffer from a fatty liver disease, said method comprising
providing a plasma or liver sample derived from a subject diagnosed to suffer from a fatty liver disease,
determining in the plasma or liver sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma or liver triglyceride (TAG) biomarker with a reference, wherein increase of TAG(52:2), TAG(54:3), TAG(56:3), TAG(50:2) or a combination and/or decrease of TAG(52:5), TAG(52:4), TAG(54:6) or a combination thereof is indicative for the fatty liver disease progression.

According to a further aspect, the present invention provides a method for monitoring the progression of a fatty liver disease in a subject diagnosed to suffer from a fatty liver disease, said method comprising
providing a plasma sample derived from a subject diagnosed to suffer from a fatty liver disease,
determining in the plasma sample of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma triglyceride (TAG) biomarker with a reference, wherein increase of TAG(52:2), TAG(54:3), TAG(56:3), TAG(50:2) or a combination and/or decrease of TAG(52:5), TAG(52:4), TAG(54:6) or a combination thereof is indicative for the fatty liver disease progression.

In the context of the present invention, the reference is derived from a sample of a subject or group of subjects known not to suffer from the fatty liver disease or the predisposition therefor. In a preferred embodiment, the sample is selected from the group comprising liver biopsy (sample), plasma sample or blood cells sample. In most preferred embodiment, the sample is selected from the group comprising liver biopsy (sample) and plasma sample.

According to an embodiment of the methods of the present invention, the reference could be from a subject or group of subjects known not to suffer from the fatty liver disease or condition, preferably, an apparently healthy subject. In such a case, an amount of the at least one triglyceride (TAG) biomarker, and/or at least one cardiolipin (CL) biomarker and/or at least one monolysocardiolipin (MLCL) biomarker found in the test sample being altered with respect to the reference is indicative for the presence of the fatty liver disease.

According to another embodiment of the methods of the present invention, the reference is derived from a sample of a subject or group of subjects known not to suffer from the fatty liver disease or the predisposition therefor or is a calculated reference. Preferably, said calculated reference is calculated from such a group of subjects. Preferably, the at least one triglyceride (TAG) biomarker is selected from the group comprising TAG(52:2), TAG(54:3), TAG(56:3), TAG(50:2), TAG(52:5), TAG(52:4), TAG(54:6) or a combination thereof, and wherein TAG(52:2), TAG(54:3), TAG(56:3) and TAG(50:2) are biomarkers of an unhealthy liver and are increased in the sample (liver, plasma and/or blood cells) in the presence of a fatty liver or the predisposition therefor, while TAG(52:5), TAG(52:4) and TAG(54:6) are biomarkers of a healthy liver and are decreased in the presence of a fatty liver or the predisposition therefor. Preferably, the at least one cardiolipin (CL) biomarker and/or monolysocardiolipin (MLCL) biomarker is selected from the group comprising CL(LLLL), MLCL(LLL), MLCL(LOO), MLCL(LLO), CL(LOOPo), CL(LLPoP), CL(LOOO), CL(OOOP) or a combination thereof, and wherein MLCL(LOO), MLCL(LLO), CL(LOOPo), CL(LLPoP), CL(LOOO) and CL(OOOP) are biomarkers of an unhealthy liver and are increased in the sample (liver, plasma, and/or blood cells) in the presence of a fatty liver or the predisposition therefor, while CL(LLLL) and MLCL(LLL) are biomarkers of a healthy liver and are decreased in the presence of a fatty liver or the predisposition therefor.

Typically, TAG(52:2) is triacylglycerol 52:2, a triglyceride in which the three acyl groups contain a total of 52 carbons and 2 double bonds; TAG(54:3) is triacylglycerol 54:3, a triglyceride in which the three acyl groups contain a total of 54 carbons and 3 double bonds; TAG(56:3) is triacylglycerol 56:3, a triglyceride in which the three acyl groups contain a total of 56 carbons and 3 double bonds; TAG(50:2) is triacylglycerol 50:2, a triglyceride in which the three acyl groups contain a total of 50 carbons and 2 double bonds; TAG(52:5) is triacylglycerol 52:5, a triglyceride in which the three acyl groups contain a total of 52 carbons and 5 double bonds; TAG(52:4) is triacylglycerol 52:4, a triglyceride in which the three acyl groups contain a total of 52 carbons and 4 double bonds; TAG(54:6) is triacylglycerol 54:6, a triglyceride in which the three acyl groups contain a total of 54 carbons and 6 double bonds.

The abbreviations are as follows: CL(LLLL) is cardiolipin-(18:2/18:2/18:2/18:2); MLCL(LLL) is monolysocardiolipin-(18:2/18:2/18:2); MLCL(LOO) is monolysocardiolipin-(18:2/18:1/18:1); MLCL(LLO) is monolysocardiolipin-(18:2/18:2/18:1); CL(LOOPo) is cardiolipin-(18:2/18:1/18:1/16:1); CL(LLPoP) is cardiolipin-(18:2/18:2/16:1/16:0); CL(LOOO) is cardiolipin-(18:2/18:1/18:1/18:1); CL(OOOP) is cardiolipin-(18:1/18:1/18:1/16:0). Cardiolipin has four alkyl chains, thus for example the feature 18:2 in CL(18:2/18:2/18:2/18:2) means 18-carbon fatty alkyl chains with 2 unsaturated bonds on each of them.

Determination of the amount of triglyceride (TAG) biomarker, cardiolipin (CL) biomarker and monolysocardiolipin (MLCL) biomarker in samples can be done by any suitable method known in the art.

According to a further aspect, the present invention provides a method for monitoring and/or adapting the efficacy of a therapy for treatment of a fatty liver disease in a subject diagnosed to suffer from a fatty liver disease, said method comprising
a) providing samples derived from a subject before and after the subject undergoes the therapy,
b) determining in the samples of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
c) comparing the amount of the at least one plasma or liver triglyceride (TAG) biomarker, liver cardiolipin (CL) biomarker and/or monolysocardiolipin (MLCL) biomarker after therapy against the amount before therapy, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for inefficacy of the therapy,
d) adapting and/or changing the therapy for treatment of a fatty liver disease in a subject if the therapy is found to be ineffective according to the step c).

In a preferred embodiment, the sample is selected from the group comprising liver biopsy (sample), plasma sample or blood cells sample. In most preferred embodiment, the sample is selected from the group comprising liver biopsy (sample) and plasma sample.

According to another aspect, the present invention provides a method for monitoring and/or adapting the efficacy of a therapy for treatment of a fatty liver disease in a subject diagnosed to suffer from a fatty liver disease, said method comprising

a) providing plasma or liver samples derived from a subject before and after the subject undergoes the therapy,
b) determining in the plasma or liver samples of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof,
c) comparing the amount of the at least one plasma or liver triglyceride (TAG) biomarker after therapy against the amount before therapy, wherein increase of TAG(52:2), TAG(54:3), TAG(56:3), TAG(50:2) or a combination thereof and/or decrease of TAG(52:5), TAG(52:4), TAG(54:6) or a combination thereof is indicative for inefficacy of the therapy,
d) adapting and/or changing the therapy for treatment of a fatty liver disease in a subject if the therapy is found to be ineffective according to the step c).

According to another aspect, the present invention provides a method for monitoring and/or adapting the efficacy of a therapy for treatment of a fatty liver disease in a subject diagnosed to suffer from a fatty liver disease, said method comprising
a) providing plasma samples derived from a subject before and after the subject undergoes the therapy,
b) determining in the plasma samples of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
c) comparing the amount of the at least one plasma triglyceride (TAG) biomarker after therapy against the amount before therapy, wherein increase of TAG(52:2), TAG(54:3), TAG(56:3), TAG(50:2) or a combination thereof and/or decrease of TAG(52:5), TAG(52:4), TAG(54:6) or a combination thereof is indicative for inefficacy of the therapy,
d) adapting and/or changing the therapy for treatment of a fatty liver disease in a subject if the therapy is found to be ineffective according to the step c).

According to another aspect, the present invention provides a method for treating a subject suffering from a fatty liver disease by a therapy, wherein the subject exhibits increased amount of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decreased amount of TAG(52:5), TAG(52:4), TAG(54:6), CL(LLLL), MLCL(LLL) or a combination thereof.

In a preferred embodiment, a therapy for treatment of a fatty liver disease is selected from the group comprising surgery, drug treatment or life style recommendations. Drug treatment comprises the administration of one or more drugs selected from Statins, Incretin analogues, Metformin, Rimonabant, Thiazolidinediones, or Orlistat. Life style recommendations typically include adapted diet plan (such as adapted food, adapted food quantites and adapted water intake) and exercise (sport) known in the art and able to have beneficial effects to treat a fatty liver disease.

In accordance with the methods of the present invention, it is possible to select one, two, three, four, five, six or seven triglyceride (TAG) biomarkers selected from the group comprising TAG(52:2), TAG(54:3), TAG(56:3), TAG(50:2), TAG(52:5), TAG(52:4), TAG(54:6) and/or one, two, three, four or five cardiolipin (CL) biomarkers selected from the group comprising CL(LLLL), CL(LOOPo), CL(LLPoP), CL(LOOO), CL(OOOP), and/or one, two or three monolysocardiolipin (MLCL) biomarkers selected from the group comprising MLCL(LLL), MLCL(LOO), MLCL(LLO) to be used in the methods of the present invention.

In an embodiment of the methods of the present invention, the fatty liver disease is selected from the group comprising non-alcoholic fatty liver disease (NAFLD), alcoholic fatty liver disease (AFLD), non-alcoholic steatohepatitis (NASH), alcoholic steatohepatitis (ASH), steatosis, other diseases that are typified by liver fat accumulation, diseases comprising genetic mutations of PNPLA3, FDFT1, GCKR, NCAN, PPP1R3B and LYPLAL1, a disease related to any genetic mutation, which confer susceptibility or predisposition to fatty liver, the metabolic syndrome including obesity, diabetes, hyperlipidemia, hypertension, and atherosclerosis, condition related to rapid weight loss and weight loss following bariatric surgery.

In a preferred embodiment of the methods of the present invention, the fatty liver disease is selected from the group comprising non-alcoholic fatty liver disease (NAFLD), alcoholic fatty liver disease (AFLD), non-alcoholic steatohepatitis (NASH), alcoholic steatohepatitis (ASH), steatosis, other diseases that are typified by liver fat accumulation.

In a more preferred embodiment of the methods of the present invention, the fatty liver disease is selected from the group comprising non-alcoholic fatty liver disease (NAFLD), alcoholic fatty liver disease (AFLD), non-alcoholic steatohepatitis (NASH), alcoholic steatohepatitis (ASH), steatosis.

In another preferred embodiment of the methods of the present invention, the fatty liver disease is selected from the group of diseases comprising genetic mutations of PNPLA3, FDFT1, GCKR, NCAN, PPP1R3B and LYPLAL1.

In a further preferred embodiment of the methods of the present invention, the fatty liver disease is selected from the group comprising a disease related to any genetic mutation, which confer susceptibility or predisposition to fatty liver.

In a further preferred embodiment of the methods of the present invention, the fatty liver disease is selected from the group comprising the metabolic syndrome including obesity, diabetes, hyperlipidemia, hypertension, and atherosclerosis.

In a further preferred embodiment of the methods of the present invention, the fatty liver disease is selected from the group comprising condition related to rapid weight loss and weight loss following bariatric surgery.

In a preferred embodiment of the methods of the present invention, the fatty liver disease is selected from the group comprising toxic hepatitis induced by drugs, such as pain relievers (acetaminophen, aspirin, ibuprofen and naproxen), herbs and supplements (aloe vera, black cohosh, cascara, chaparral, comfrey, kava and ephedra); industrial chemicals (carbon tetrachloride, vinyl chloride, paraquat and polychlorinated biphenyls).

According to a further aspect, the present invention provides use of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof, for in-vitro diagnosing fatty liver disease or the predisposition therefor.

According to a further aspect, the present invention provides use of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof for in-vitro diagnosing fatty liver disease or the predisposition therefor.

According to a further aspect, the present invention provides use of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof for in-vitro diagnosing fatty liver disease or the predisposition therefor.

According to further aspect, the present invention provides a plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof for use in diagnosing fatty liver disease or the predisposition therefor in a subject.

According to another aspect, the present invention provides a plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof for use in diagnosing fatty liver disease or the predisposition therefor in a subject.

According to another aspect, the present invention provides a plasma triglyceride (TAG) biomarker selected from the group comprising TAG(52:2), TAG(54:3), TAG(56:3), TAG(50:2), TAG(52:5), TAG(52:4), TAG(54:6) or a combination thereof for use in diagnosing fatty liver disease or the predisposition therefor in a subject.

Advantageously, it has been found in the present invention that the amounts of the specific triglyceride biomarkers, cardiolipin (CL) biomarkers and monolysocardiolipin (MLCL) biomarkers referred to above are indicators for a fatty liver disease or a predisposition therefor. Thus fatty liver diseases can be safely, simply, not-invasively, efficiently and reliably diagnosed by diagnosis based on body fluids such as plasma samples. An analysis via a body fluid metabolite profile is significantly less cost intensive than the gold standard liver biopsy with subsequent histopathology. This is particularly helpful for an efficient diagnosis of the disease as well as for improving of the pre-clinical and clinical management of a fatty liver disease as well as an efficient monitoring of patients. Moreover, based on the methods according to the present invention, the development of therapeutic measures including drugs can be facilitated and guided. Further, therapies or life style recommendations which are applied can be easily monitored for success without taking a serious risk of adverse side effects caused by the monitoring method.

In light of the foregoing, the methods of the present invention can be used for i) monitoring a subject suffering from a fatty liver disease, i.e. disease progression or amelioration can be determined, ii) identifying a subject in need of a therapy of a fatty liver disease, iii) identifying whether a therapy against a fatty liver disease is successful in a subject.

The present invention, therefore, also relates to a method for identifying whether a subject is in need for a therapy of a fatty liver disease comprising the steps of the methods of the present invention and the further step of identifying a subject in need if the fatty liver disease is diagnosed.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated, and not limiting, the scope of the invention being indicated by the appended Claims, and all changes which come within the meaning and range of equivalency are intended to be embraced therein. The foregoing description will be more fully understood with reference to the following examples. Such Examples, are, however, exemplary of methods of practising the present invention and are not intended to limit the scope of the invention.

### EXAMPLES

Animals

50 strains of the BXD population were phenotyped. ˜10 male mice from each strain were separated at 8 weeks of age into two equal groups for each dietary cohort. Mice were fed a chow diet (Harlan Teklad Global 18% Protein Rodent Diet, 2018, 6% kcal/fat) after weaning at 3 weeks of age until 8 weeks of age. At 8 weeks of age, half the population switched to a HFD (Harlan Teklad, TD.06414, 60% kcal/fat) for 21 weeks. All mice were phenotyped as previously described (Williams et al., 2016). At week 29, animals were fasted overnight then anesthetised using isoflurane. After blood collection, perfusion was performed via vena cava and ˜23 organs including liver were collected. Due to insufficient plasma (used for plasma biochemistry), lipidomics analysis could be performed only on 44 CD and 34 HFD strains. All research was approved by the Swiss cantonal veterinary authorities of Vaud under licenses 2257 and 2257.1.

Lipids were normalized in three different ways—normalized to internal standards (of each class), to total lipids in each sample and to all lipids in each class. Basic quality check and QTL analysis was performed from all the datasets, however the dataset normalized to total lipids was used for all the analysis due to overall low relative standard deviation in this dataset (data not shown). Additionally, normalization to total lipids has two major advantages over the other normalization methods; 1) All lipids measured did not have a true internal standard, 2) for lipid classes that have few lipid species measured, normalizing to class will be largely driven by one or two highly abundant lipids.

Validation of lipid species as biomarkers of fatty liver and metabolic fitness in mouse and human subjects were analysed (data not shown). For mice: n=7-10 per group. For human subjects: n=12, healthy; n=7 simple steatosis (F0=no fibrosis); n=7 NASH, fibrosis grade 1; n=8 NASH, fibrosis grade 2; n=7 NASH, fibrosis grade 3; n=5 NASH, cirrhosis=fibrosis grade 4. Groups with fibrosis grade 1 and 2 were combined as one group (low grade fibrosis), whereas groups with fibrosis grade 3 and 4 were combined as one group (advance fibrosis).

Bioinformatic Analyses

Correlations are Pearson's r or Spearman's rho, depending on the distribution of data. Data normality were checked by the Shapiro-Wilk test in R, with a W≥0.90 considered “normal”. Student's t-test was used for two groups comparisons in normal data of equal varainces, and Welch's t-test otherwise. QTL calculations were performed using the R/qtl (v 1.39-5) package (Arends et al., 2010) on the log transformed data. Parametric QTL calculation was performed for normally distributed lipids and non-parametric for lipids, those were not normally distributed. QTLs with a LOD score >3.5 were considered locally significant (local p<0.05) while QTLs with a LOD score >3.5 were considered genome-wide significant (genome-wide p<0.05).

Biological enrichment analysis was performed using the WEB-based Enrichr (http://amp.pharm.mssm.edu/Enrichr/). Heatmaps were generated using the “heatmap.2” function in R. PCA analysis was performed using “prcomp” function in R. Unweighted correlation network graphs were performed using Spearman correlation, keeping all edges with P values less than 1e-05 in both D and HFD in R using the custom package imsblnfer, currently on Github (https://github.com/wolski/imsbInfer) but in the process of being added to Bioconductor. All graphs and analyses were either in R or GraphPad. For R, standard R plotting packages included in gplots or ggplot2—e.g., stripchart, plotCI, and barplot2. Final figures were all prepared with Adobe Illustrator.

Weighted gene correlation network analysis Weighted gene correlation network analysis (WGCNA) was performed as previously described (Langfelder and Horvath, 2008) by using the WGCNA R software package (v1.51). To construct the weighed lipid coexpression network, a correlation matrix was calculated containing all pairwise Pearson's correlations between all pairs of lipids across all BXD strains for both CD and HFD. It was defined a “signed hybrid” network in which the adjacency takes values between 0 and 1 when the correlation is positive and 0 if the correlation is negative. A power of 26 was chosen for both CD and HFD datasets. It was selected the minimum power in which both datasets followed the Scale-Free Topology Criterion (model fitting index R²>0.8) and showed a similar connectivity. The selection of a high power (threshold) has the effect of suppressing low correlations that may be due to noise, penalize weaker connections and strengthen stronger connections. The result is a network adjacency that is zero for negatively correlated genes and is positive for positively correlated genes. Adjacency of weakly correlated genes is nearly zero due to the power transformation. Next, the lipids were hierarchically clustered using the distance measure and modules were determined by choosing a height cutoff for the resulting dendrogram by using a dynamic tree-cutting algorithm, selecting a minimum module size of 5. Modules with a correlation higher than 0.75 were merged. The resulting lipid modules were assigned color names and identified using the eigenvector of each module, named as module eigenlipid (ME). ME is defined as the first principal component of the standardized expression profiles and can be considered as the best summary of the standardized module expression data. Correspondence analysis between CD and HFD was performed by calculating the overlaps of each pair of CD-HFD modules and analyzed using the Fisher's exact test. Module-Trait relationships were calculated by Pearson's correlation between MEs and selected metabolic phenotypic variables obtained in previous BXD studies [ref] in order to identify modules related to metabolic traits. Module QTL (modQTL) mapping were calculated by selecting the MEs as phenotype traits using the R package R/qtl (v 1.39-5) (Broman et al., 2003).

Methods Corresponding to FIG. 12

Human Plasma Biochemistry

Fasting whole blood samples were obtained by venipuncture after an overnight fast of 8 hours or more and processed for plasma within 2 hours on the day of liver biopsy. Routine blood tests were performed and included measures of ALT, AST, fasting TAG, cholesterol, glucose and insulin. The clinical information of the participants is provided in the table 3 below.

| | | | NASH, | NASH, | |
|---|---|---|---|---|---|
| | Healthy | Steatosis | early stage | Adv. stage | p-value |
| Age (years) | 27.83 ± 1.64 | 42.14 ± 3.29 | 46.43 ± 3.77 | 58.18 ± 2.78 | a, b, c, e, f |
| Body weight | 65.4 ± 1.92 | 105.14 ± 10.63 | 94.92 ± 4.79 | 111.64 ± 8.90 | a, b, c |
| BMI (Kg/m²) | 21.89 ± 0.50 | 33.91 ± 3.21 | 32.37 ± 1.63 | 36.95 ± 2.79 | a, b, c |
| Fibroscan (kPa) | 4.28 ± 0.28 | 9.03 ± 2.41 | 8.81 ± 1.21 | 19.58 ± 2.88 | c, e, f |
| Steatosis (%) | — | 38.57 ± 7.46 | 64.64 ± 5.70 | 45.45 ± 8.08 | d |
| NAS score | — | 3.29 ± 0.47 | 4.93 ± 0.29 | 4.64 ± 0.48 | d |
| ALT (U/L) | 18.92 ± 1.43 | 50.14 ± 6.97 | 84.79 ± 15.34 | 70 ± 10.98 | b, c |
| AST (U/L) | 22.50 ± 1.31 | 30.29 ± 2.73 | 44.79 ± 5.14 | 72.82 ± 27.52 | c |
| Cholesterol | 173 ± 11.39 | 204.71 ± 25.37 | 179.21 ± 9.82 | 173.09 ± 9.76 | |
| (mg/dl) | | | | | |
| Triglyceride | 73.25 ± 7.71 | 190.43 ± 36.18 | 167.14 ± 17.21 | 140.82 ± 2 | a, b |
| (mg/dl) | | | | | |
| Fasting glucose | 84.08 ± 2.30 | 100.14 ± 6.60 | 138.43 ± 15.81 | 120.18 ± 9.53 | b |
| (mg/dl) | | | | | |
| Fasting insulin | 6.70 ± 1.11 | 24.33 ± 10.06 | 19.34 ± 1.91 | 23.72 ± 3.16 | a, c |
| (μU/mL) | | | | | |

Subjects: n = 44; 12 healthy, 7 steatosis, 14 early stage NASH, 11 adv. Stage NASH
Values of mean ± SEM are represented.
*p < 0.05 from one-way ANOVA with Tukey's multiple comparison test correction.
Significant difference between groups are indicated as:
“a” between Healthy and Steatosis
“b” between Healthy and NASH, early stage
“c” between Healthy and NASH, Adv. Stage
“d” between Steatosis and NASH, early stage
“e” between Steatosis and NASH, Adv. Stage
“f” between NASH, early stage and NASH, Adv. stage

Methods Corresponding to FIG. 13

Lipidomics Sample Preparation and Analysis

Internal Standards (IS) Used:

For BXD liver samples, Q6, PC(15:0/15:0), PS(17:0/17:0), PE (15:0/15:0), PA (17:0/17:0), PG (15:0/15:0), CL (56:0) and FA (15:0/15:0) were used as internal standards. For NAFLD signature validation experiments in mice (FIG. 2), the standard mix SPLASH® Lipidomix® Mass Spec Standard | 330707, supplemented with Q6 and CL(56:0) were used.

Extractions:

Liver samples were weighed and homogenized using a Potter-Elvehjem tissue grinder in 1.5 mL homogenization buffer (8 M urea, 50 mM TEAB, 100 mM NaCl, 1 mM CaCI, protease and phosphatase inhibitors (Roche)). Protein concentration was determined by BCA and all samples were diluted to 8 mg/mL with homogenization buffer. Samples were aliquoted with each tube containing 1 mg of protein (125 μL) and flash frozen in liquid N₂ and stored at −80° C. Frozen aliquots of liver extracts were thawed on ice then internal standards were added (20 μL) and samples were vortexed (30 s). Chloroform/methanol (1:1, v/v, 1000 μL) was added and samples were vortexed (60 s). Subsequently, HCl (1 M, 200 μL) was added to induce phase separation, followed by vortex (60 s) and centrifugation (3,000 g, 3 min, 4° C.) to complete phase separation. 550 μL of the organic phase was dried under Ar₂(g). The organic residue was reconstituted in ACN/IPA/H₂O (65:30:5, v/v/v, 100 μL) by vortexing (60 s) and transferred to a glass vial for LC-MS analysis. Samples were stored at ˜80° C. until further use. LC-MS analysis was performed on an Ascentis Express C18 column held at 50° C. (150 mm×2.1 mm×2.7 μm particle size; Supelco) using an Accela LC Pump (500 μL/min flow rate; Thermo). Mobile phase A consisted of 10 mM ammonium acetate in ACN/H₂O (70:30, v/v) containing 250 μL/L acetic acid. Mobile phase B consisted of 10 mM ammonium acetate in IPA/ACN (90:10, v/v) with the same additives. 10 μL of sample were injected by an HTC PAL autosampler (Thermo). Initially, mobile phase B was held at 40% for 30 s and then increased to 50% over an additional 30 s. It was then increased to 55% over 4 min after which, it was increased to 99% over 6 min and held there for 3 min. Prior to the next injection, the column was reequilibrated for 2 min. The LC system was coupled to a Q Exactive mass spectrometer (Build 2.3 SP2) by a HESI II heated ESI source kept at 325° C. (Thermo). The inlet capillary was kept at 320° C., sheath gas was set to 35 units, auxiliary gas to 15 units, and the spray voltage was set to 3,000 V in negative mode and 4,000 V in positive mode.

Several scan functions, including targeted and untargeted, were used to ensure optimal data acquisition for each lipid class. For fatty acids, selected ion monitoring (SIM) scans were taken from 0-3 min. MS¹ data was acquired in negative mode for 220-600 m/z at a resolving power of 17,500 and an AGC target of 1×10⁵. For phospholipids, diacylglycerols, and CoQ, parallel reaction monitoring (PRM) was used. The instrument was run in negative mode with a resolving power of 17,500, an AGC target of 2×10⁵, a maximum injection time of 75 ms, and isolation window of 1.2 Th. Scans targeting each species were scheduled between 0-10.3 min based on previously determined retention times. For triglycerides, a separate set of runs was done where in addition to the targeted method described above, the MS was operated in positive mode from 10.1-13 min with resolving power set at 17,500 and the AGC target set to 5×10⁵. Ions from 750-1,100 m/z were isolated (Top 2) and fragmented.

Measurement, Normalization and Quality Control:

The selection of lipid species for measurement was based on their abundance, stability, polarity and ease of ionization. For the BXDs, peaks were automatically integrated using TraceFinder software (Thermo) and integrations were checked manually. NR mice were processed as described in Jha et al., 2018. Lipids were normalized in three different ways—to internal standards (of each class), to total lipids in each sample and to all lipids in each class. Basic quality check and QTL analysis was performed from all the datasets, however the dataset normalized to total lipids was used for all the analysis and figures shown in the manuscript due to the overall low relative standard deviation in this dataset (data not shown). Additionally, normalization to total lipids has two major advantages over the other normalization methods; 1) All lipids measured did not have a true internal standard, 2) for lipid classes that have few lipid species measured, normalizing to class will be largely driven by one or two highly abundant lipids. Quality assessment of the MS measurements was performed by comparing the reproducibility of the technical and extraction replicates (see FIG. 6A-6C). Note: lipid pairs marked with “_1” and “_2” (TAGs 54:5, 54:6, 56:7; PI(Dha/S) and CL(LLOPo) indicate two isobaric peaks. The TAGs are isobaric peaks with different fatty acid compositions while the PI and CL are isobaric because they have the exact same fatty acid composition but are likely ordered differently to cause chromatographic separation.

### REFERENCES

Anjani, K., Lhomme, M., Sokolovska, N., Poitou, C., Aron-Wisnewsky, J., Bouillot, J. L., Lesnik, P., Bedossa, P., Kontush, A., Clement, K., et al. (2015). Circulating phospholipid profiling identifies portal contribution to NASH signature in obesity. Journal of hepatology 62, 905-912.
Arends, D., Prins, P., Jansen, R. C., and Broman, K. W. (2010). R/qtl: high-throughput multiple QTL mapping. Bioinformatics 26, 2990-2992.
Broman, K. W., Wu, H., Sen, S., and Churchill, G. A. (2003). R/qtl: QTL mapping in experimental crosses. Bioinformatics 19, 889-890.
Browning, J. D., Szczepaniak, L. S., Dobbins, R., Nuremberg, P., Horton, J. D., Cohen, J. C., Grundy, S. M., and Hobbs, H. H. (2004). Prevalence of hepatic steatosis in an urban population in the United States: impact of ethnicity. Hepatology 40, 1387-1395.
Burke, D. T., Kozloff, K. M., Chen, S., West, J. L., Wilkowski, J. M., Goldstein, S. A., Miller, R. A., and Galecki, A. T. (2012). Dissection of complex adult traits in a mouse synthetic population. Genome research 22, 1549-1557.
Chalasani, N., Younossi, Z., Lavine, J. E., Diehl, A. M., Brunt, E. M., Cusi, K., Charlton, M., and Sanyal, A. J. (2012). The diagnosis and management of non-alcoholic fatty liver disease: practice Guideline by the American Association for the Study of Liver Diseases, American College of Gastroenterology, and the American Gastroenterological Association. Hepatology 55, 2005-2023.
Champy, M. F., Selloum, M., Piard, L., Zeitler, V., Caradec, C., Chambon, P., and Auwerx, J. (2004). Mouse functional genomics requires standardization of mouse handling and housing conditions. Mamm Genome 15, 768-783.
Chick, J. M., Munger, S. C., Simecek, P., Huttlin, E. L., Choi, K., Gatti, D. M., Raghupathy, N., Svenson, K. L., Churchill, G. A., and Gygi, S. P. (2016). Defining the consequences of genetic variation on a proteome-wide scale. Nature 534, 500-505.
Chitraju, C., Trotzmuller, M., Hartler, J., Wolinski, H., Thallinger, G. G., Lass, A., Zechner, R., Zimmermann, R., Kofeler, H. C., and Spener, F. (2012). Lipidomic analysis of lipid droplets from murine hepatocytes reveals distinct signatures for nutritional stress. Journal of lipid research 53, 2141-2152.
Cohen, J. C., Horton, J. D., and Hobbs, H. H. (2011). Human fatty liver disease: old questions and new insights. Science 332, 1519-1523.
Consortium, G. T. (2015). Human genomics. The Genotype-Tissue Expression (GTEx) pilot analysis: multitissue gene regulation in humans. Science 348, 648-660.
Diabetes Genetics Initiative of Broad Institute of, H., Mit, L. U., Novartis Institutes of BioMedical, R., Saxena, R., Voight, B. F., Lyssenko, V., Burtt, N. P., de Bakker, P. I., Chen, H., Roix, J. J., et al. (2007). Genome-wide association analysis identifies loci for type 2 diabetes and triglyceride levels. Science 316, 1331-1336.
Fahy, E., Subramaniam, S., Brown, H. A., Glass, C. K., Merrill, A. H., Jr., Murphy, R. C., Raetz, C. R., Russell, D. W., Seyama, Y., Shaw, W., et al. (2005). A comprehensive classification system for lipids. Journal of lipid research 46, 839-861.

Farese, R. V., Jr., Zechner, R., Newgard, C. B., and Walther, T. C. (2012). The problem of establishing relationships between hepatic steatosis and hepatic insulin resistance. Cell Metab 15, 570-573.
Gariani, K., Menzies, K. J., Ryu, D., Wegner, C. J., Wang, X., Ropelle, E. R., Moullan, N., Zhang, H., Perino, A., Lemos, V., et al. (2016). Eliciting the mitochondrial unfolded protein response by nicotinamide adenine dinucleotide repletion reverses fatty liver disease in mice. Hepatology 63, 1190-1204.
Gieger, C., Geistlinger, L., Altmaier, E., Hrabe de Angelis, M., Kronenberg, F., Meitinger, T., Mewes, H. W., Wichmann, H. E., Weinberger, K. M., Adamski, J., et al. (2008). Genetics meets metabolomics: a genome-wide association study of metabolite profiles in human serum. PLoS Genet 4, e1000282.
Global Lipids Genetics, C., Willer, C. J., Schmidt, E. M., Sengupta, S., Peloso, G. M., Gustafsson, S., Kanoni, S., Ganna, A., Chen, J., Buchkovich, M. L., et al. (2013). Discovery and refinement of loci associated with lipid levels. Nat Genet 45, 1274-1283.
Gorden, D. L., Myers, D. S., Ivanova, P. T., Fahy, E., Maurya, M. R., Gupta, S., Min, J., Spann, N. J., McDonald, J. G., Kelly, S. L., et al. (2015). Biomarkers of NAFLD progression: a lipidomics approach to an epidemic. Journal of lipid research 56, 722-736.
Han, X. (2016). Lipidomics for studying metabolism. Nature reviews Endocrinology 12, 668-679.
Hui, S. T., Parks, B. W., Org, E., Norheim, F., Che, N., Pan, C., Castellani, L. W., Charugundla, S., Dirks, D. L., Psychogios, N., et al. (2015). The genetic architecture of NAFLD among inbred strains of mice. eLife 4, e05607.
Hyotylainen, T., and Oresic, M. (2014). Systems biology strategies to study lipidomes in health and disease. Progress in lipid research 55, 43-60.
Hyotylainen, T., and Oresic, M. (2015). Analytical Lipidomics in Metabolic and Clinical Research. Trends in endocrinology and metabolism: TEM 26, 671-673.
Hyysalo, J., Gopalacharyulu, P., Bian, H., Hyotylainen, T., Leivonen, M., Jaser, N., Juuti, A., Honka, M. J., Nuutila, P., Olkkonen, V. M., et al. (2014). Circulating triacylglycerol signatures in nonalcoholic fatty liver disease associated with the I148M variant in PNPLA3 and with obesity. Diabetes 63, 312-322.
Illig, T., Gieger, C., Zhai, G., Romisch-Margl, W., Wang-Sattler, R., Prehn, C., Altmaier, E., Kastenmuller, G., Kato, B. S., Mewes, H. W., et al. (2010). A genome-wide perspective of genetic variation in human metabolism. Nat Genet 42, 137-141.
Johansen, C. T., Kathiresan, S., and Hegele, R. A. (2011). Genetic determinants of plasma triglycerides. Journal of lipid research 52, 189-206.
Kathiresan, S., Willer, C. J., Peloso, G. M., Demissie, S., Musunuru, K., Schadt, E. E., Kaplan, L., Bennett, D., Li, Y., Tanaka, T., et al. (2009). Common variants at 30 loci contribute to polygenic dyslipidemia. Nat Genet 41, 56-65.
Kirk, E. A., Moe, G. L., Caldwell, M. T., Lernmark, J. A., Wilson, D. L., and LeBoeuf, R. C. (1995). Hyper- and hypo-responsiveness to dietary fat and cholesterol among inbred mice: searching for level and variability genes. Journal of lipid research 36, 1522-1532.
Langfelder, P., and Horvath, S. (2008). WGCNA: an R package for weighted correlation network analysis. BMC bioinformatics 9, 559.
Li, Z., Agellon, L. B., Allen, T. M., Umeda, M., Jewell, L., Mason, A., and Vance, D. E. (2006). The ratio of phosphatidylcholine to phosphatidylethanolamine influences membrane integrity and steatohepatitis. Cell Metab 3, 321-331.
Lin, X., Yue, P., Chen, Z., and Schonfeld, G. (2005). Hepatic triglyceride contents are genetically determined in mice: results of a strain survey. American journal of physiology Gastrointestinal and liver physiology 288, G1179-1189.
Listenberger, L. L., Han, X., Lewis, S. E., Cases, S., Farese, R. V., Jr., Ory, D. S., and Schaffer, J. E. (2003). Triglyceride accumulation protects against fatty acid-induced lipotoxicity. Proceedings of the National Academy of Sciences of the United States of America 100, 3077-3082.

Ma, D. W., Arendt, B. M., Hillyer, L. M., Fung, S. K., McGilvray, I., Guindi, M., and Allard, J. P. (2016). Plasma phospholipids and fatty acid composition differ between liver biopsy-proven nonalcoholic fatty liver disease and healthy subjects. Nutrition & diabetes 6, e220.
Manolio, T. A., Collins, F. S., Cox, N. J., Goldstein, D. B., Hindorff, L. A., Hunter, D. J., McCarthy, M. I., Ramos, E. M., Cardon, L. R., Chakravarti, A., et al. (2009). Finding the missing heritability of complex diseases. Nature 461, 747-753.
Oresic, M., Hyotylainen, T., Kotronen, A., Gopalacharyulu, P., Nygren, H., Arola, J., Castillo, S., Mattila, I., Hakkarainen, A., Borra, R. J., et al. (2013). Prediction of non-alcoholic fatty-liver disease and liver fat content by serum molecular lipids. Diabetologia 56, 2266-2274.
Puri, P., Baillie, R. A., Wiest, M. M., Mirshahi, F., Choudhury, J., Cheung, O., Sargeant, C., Contos, M. J., and Sanyal, A. J. (2007). A lipidomic analysis of nonalcoholic fatty liver disease. Hepatology 46, 1081-1090.
Puri, P., Wiest, M. M., Cheung, O., Mirshahi, F., Sargeant, C., Min, H. K., Contos, M. J., Sterling, R. K., Fuchs, M., Zhou, H., et al. (2009). The plasma lipidomic signature of nonalcoholic steatohepatitis. Hepatology 50, 1827-1838.
Romeo, S., Kozlitina, J., Xing, C., Pertsemlidis, A., Cox, D., Pennacchio, L. A., Boerwinkle, E., Cohen, J. C., and Hobbs, H. H. (2008). Genetic variation in PNPLA3 confers susceptibility to nonalcoholic fatty liver disease. Nat Genet 40, 1461-1465.
Shin, S. Y., Fauman, E. B., Petersen, A. K., Krumsiek, J., Santos, R., Huang, J., Arnold, M., Erte, I., Forgetta, V., Yang, T. P., et al. (2014). An atlas of genetic influences on human blood metabolites. Nat Genet 46, 543-550.
Sittig, L. J., Carbonetto, P., Engel, K. A., Krauss, K. S., Barrios-Camacho, C. M., and Palmer, A. A. (2016). Genetic Background Limits Generalizability of Genotype-Phenotype Relationships. Neuron 91, 1253-1259.
Speliotes, E. K., Yerges-Armstrong, L. M., Wu, J., Hernaez, R., Kim, L. J., Palmer, C. D., Gudnason, V., Eiriksdottir, G., Garcia, M. E., Launer, L. J., et al. (2011). Genome-wide association analysis identifies variants associated with nonalcoholic fatty liver disease that have distinct effects on metabolic traits. PLoS Genet 7, e1001324.
Surakka, I., Horikoshi, M., Magi, R., Sarin, A. P., Mahajan, A., Lagou, V., Marullo, L., Ferreira, T., Miraglio, B., Timonen, S., et al. (2015). The impact of low-frequency and rare variants on lipid levels. Nat Genet 47, 589-597.
Teslovich, T. M., Musunuru, K., Smith, A. V., Edmondson, A. C., Stylianou, I. M., Koseki, M., Pirruccello, J. P., Ripatti, S., Chasman, D. I., Willer, C. J., et al. (2010). Biological, clinical and population relevance of 95 loci for blood lipids. Nature 466, 707-713.
van der Vusse, G. J., van Bilsen, M., and Glatz, J. F. (2000). Cardiac fatty acid uptake and transport in health and disease. Cardiovascular research 45, 279-293.
Williams, E. G., and Auwerx, J. (2015). The Convergence of Systems and Reductionist Approaches in Complex Trait Analysis. Cell 162, 23-32.
Williams, E. G., Wu, Y. B., Jha, P., Dubuis, S., Blattmann, P., Argmann, C. A., Houten, S. M., Amariuta, T., Wolski, W., Zamboni, N., et al. (2016). Systems proteomics of liver mitochondria function. Science 352, 1292-+.
Wu, Y., Williams, E. G., Dubuis, S., Mottis, A., Jovaisaite, V., Houten, S. M., Argmann, C. A., Faridi, P., Wolski, W., Kutalik, Z., et al. (2014). Multilayered genetic and omics dissection of mitochondrial activity in a mouse reference population. Cell 158, 1415-1430.
Zhang, W., Korstanje, R., Thaisz, J., Staedtler, F., Harttman, N., Xu, L., Feng, M., Yanas, L., Yang, H., Valdar, W., et al. (2012). Genome-wide association mapping of quantitative traits in outbred mice. G3 2, 167-174.

1. A method for diagnosing a fatty liver disease or a predisposition therefor in a subject, said method comprising
providing a sample derived from a subject suspected to suffer from a fatty liver disease,
determining in the sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
comparing the amount of the at least one plasma or liver triglyceride (TAG) biomarker, liver cardiolipin (CL) biomarker and/or monolysocardiolipin (MLCL) biomarker with a reference, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for the fatty liver disease.

2. A method for monitoring the progression of a fatty liver disease in a subject diagnosed to suffer from a fatty liver disease, said method comprising
providing a sample derived from a subject diagnosed to suffer from a fatty liver disease,
determining in the sample of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
comparing the amount of the at least one plasma or liver triglyceride (TAG) biomarker, liver cardiolipin (CL) biomarker and/or monolysocardiolipin (MLCL) biomarker with a reference, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for the fatty liver disease progression.

3. The method of claim 2, wherein the sample is selected from the group comprising liver biopsy and plasma sample.

4. The method of claim 3, wherein said method comprising
providing a plasma sample derived from a subject suspected to suffer from a fatty liver disease,
determining in the plasma sample of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma triglyceride (TAG) biomarker with a reference, wherein increase of TAG(52:2), TAG(54:3), TAG(56:3), TAG(50:2) or a combination thereof and/or decrease of TAG(52:5), TAG(52:4), TAG(54:6) or a combination thereof is indicative for the fatty liver disease.

5. The method of claim 2, wherein said method comprising
providing a plasma sample derived from a subject diagnosed to suffer from a fatty liver disease,
determining in the plasma sample of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma triglyceride (TAG) biomarker with a reference, wherein increase of TAG(52:2), TAG(54:3), TAG(56:3), TAG(50:2) or a combination and/or decrease of TAG(52:5), TAG(52:4), TAG(54:6) or a combination thereof is indicative for the fatty liver disease progression.

6. The method of claim 2, wherein the reference is derived from a sample of a subject or group of subjects known not to suffer from the fatty liver disease or the predisposition therefor.

7. A method for monitoring and/or adapting the efficacy of a therapy for treatment of a fatty liver disease in a subject diagnosed to suffer from a fatty liver disease, said method comprising
a) providing samples derived from a subject before and after the subject undergoes the therapy,
b) determining in the samples of a subject the amount of at least one plasma or liver triglyceride (TAG) biomarker selected from the group comprising TAG(52:2), TAG(54:3), TAG(56:3), TAG(50:2), TAG(52:5), TAG(52:4), TAG(54:6) or a combination thereof, and/or at least one liver cardiolipin (CL) biomarker and/or monolysocardiolipin (MLCL) biomarker selected from the group comprising CL(LLLL), MLCL(LLL), MLCL(LOO), MLCL(LLO), CL(LOOPo), CL(LLPoP), CL(LOOO), CL(OOOP) or a combination thereof,
c) comparing the amount of the at least one plasma or liver triglyceride (TAG) biomarker, liver cardiolipin (CL) biomarker and/or monolysocardiolipin (MLCL) biomarker after therapy against the amount before therapy, wherein increase of TAG(52:2), TAG(54:3), TAG(56:3), TAG(50:2), MLCL(LOO), MLCL(LLO), CL(LOOPo), CL(LLPoP), CL(LOOO), CL(OOOP) or a combination thereof and/or decrease of TAG(52:5), TAG(52:4), TAG(54:6), CL(LLLL), MLCL(LLL) or a combination thereof is indicative for inefficacy of the therapy,
d) adapting and/or changing the therapy for treatment of a fatty liver disease in a subject if the therapy is found to be ineffective according to the step c).

8. The method of claim 7, wherein the samples are selected from the group comprising liver biopsy and plasma sample.

9. The method of claim 7, wherein said method comprising
a. providing plasma samples derived from a subject before and after the subject undergoes the therapy,
b. determining in the plasma samples of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
c. comparing the amount of the at least one plasma triglyceride (TAG) biomarker after therapy against the amount before therapy, wherein increase of TAG(52:2), TAG(54:3), TAG(56:3), TAG(50:2) or a combination and/or decrease of TAG(52:5), TAG(52:4), TAG(54:6) or a combination thereof is indicative for inefficacy of the therapy,
d. adapting and/or changing the therapy for treatment of a fatty liver disease in a subject if the therapy is found to be ineffective according to the step c).

10. The method of claim 7, wherein a therapy for treatment of a fatty liver disease is selected from the group comprising surgery, drug treatment or life style recommendations.

11. The method of claim 10, wherein drug treatment comprises the administration of one or more drugs selected from the group comprising Statins, Incretin analogues, Metformin, Rimonabant, Thiazolidinediones, or Orlistat.

12. The method of claim 7, wherein the fatty liver disease is selected from the group comprising non-alcoholic fatty liver disease (NAFLD), alcoholic fatty liver disease (AFLD), non-alcoholic steatohepatitis (NASH), alcoholic steatohepatitis (ASH), steatosis, other diseases that are typified by liver fat accumulation, diseases comprising genetic mutations of PNPLA3, FDFT1, GCKR, NCAN, PPP1R3B and LYPLAL1, a disease related to any genetic mutation, which confer susceptibility or predisposition to fatty liver, the metabolic syndrome including obesity, diabetes, hyperlipidemia, hypertension, and atherosclerosis, condition related to rapid weight loss and weight loss following bariatric surgery.

13-18. (canceled)

19. The method of claim 1, wherein the sample is selected from the group comprising liver biopsy and plasma sample.

20. The method of claim 1, wherein said method comprising
providing a plasma sample derived from a subject suspected to suffer from a fatty liver disease,
determining in the plasma sample of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma triglyceride (TAG) biomarker with a reference, wherein increase of TAG(52:2), TAG(54:3), TAG(56:3), TAG(50:2) or a combination thereof and/or decrease of TAG(52:5), TAG(52:4), TAG(54:6) or a combination thereof is indicative for the fatty liver disease.

21. The method of claim 19, wherein said method comprising
providing a plasma sample derived from a subject diagnosed to suffer from a fatty liver disease,
determining in the plasma sample of a subject the amount of at least one plasma triglyceride (TAG) biomarker selected from the group comprising TAG(52:2), TAG(54:3), TAG(56:3), TAG(50:2), TAG(52:5), TAG(52:4), TAG(54:6) or a combination thereof,
comparing the amount of the at least one plasma triglyceride (TAG) biomarker with a reference, wherein increase of TAG(52:2), TAG(54:3), TAG(56:3), TAG(50:2) or a combination and/or decrease of TAG(52:5), TAG(52:4), TAG(54:6) or a combination thereof is indicative for the fatty liver disease progression.

22. The method of claim 1, wherein the reference is derived from a sample of a subject or group of subjects known not to suffer from the fatty liver disease or the predisposition therefor.

23. The method of claim 1, wherein the fatty liver disease is selected from the group comprising non-alcoholic fatty liver disease (NAFLD), alcoholic fatty liver disease (AFLD), non-alcoholic steatohepatitis (NASH), alcoholic steatohepatitis (ASH), steatosis, other diseases that are typified by liver fat accumulation, diseases comprising genetic mutations of PNPLA3, FDFT1, GCKR, NCAN, PPP1R3B and LYPLAL1, a disease related to any genetic mutation, which confer susceptibility or predisposition to fatty liver, the metabolic syndrome including obesity, diabetes, hyperlipidemia, hypertension, and atherosclerosis, condition related to rapid weight loss and weight loss following bariatric surgery.

24. The method of claim 2, wherein the fatty liver disease is selected from the group comprising non-alcoholic fatty liver disease (NAFLD), alcoholic fatty liver disease (AFLD), non-alcoholic steatohepatitis (NASH), alcoholic steatohepatitis (ASH), steatosis, other diseases that are typified by liver fat accumulation, diseases comprising genetic mutations of PNPLA3, FDFT1, GCKR, NCAN, PPP1R3B and LYPLAL1, a disease related to any genetic mutation, which confer susceptibility or predisposition to fatty liver, the metabolic syndrome including obesity, diabetes, hyperlipidemia, hypertension, and atherosclerosis, condition related to rapid weight loss and weight loss following bariatric surgery.
